(19) United States
(12) Patent Application Publication
(10) Pub. No.: US 2014/0170146 A1
(43) Pub. Date: Jun. 19, 2014

(54) METHODS FOR MULTIPLEXED DRUG EVALUATION

(71) Applicant: PRESAGE BIOSCIENCES, INC., SEATTLE, WA (US)

(72) Inventors: Richard Klinghoffer, Seattle, WA (US); Nathan Caffo, San Mateo, CA (US)

(73) Assignee: PRESAGE BIOSCIENCES, INC., SEATTLE, WA (US)

(21) Appl. No.: 14/078,977

(22) Filed: Nov. 13, 2013

Related U.S. Application Data
(60) Provisional application No. 61/725,978, filed on Nov. 13, 2012.

(51) Int. Cl. G01N 33/50; G01N 33/15

(52) U.S. Cl. CPC G01N 33/5088; G01N 33/15
USPC 424/135.1; 435/29; 435/6.1; 506/10; 435/23; 435/7.1; 435/19; 435/5; 435/15; 435/28

(57) ABSTRACT

Methods for multiplexed delivery of agents to a solid tissue in vivo followed by assessment of efficacy with mass spectrometry are described.

### CROSS-REFERENCE

This application claims the benefit of U.S. Provisional Application No. 61/725,978, filed Nov. 13, 2012, which application is incorporated herein by reference.

### BACKGROUND

Numerous cancer-related therapeutics are under preclinical, phase I or phase II clinical trial and evaluations at any particular time; however, most of them will fail to advance. In fact, numerous drug candidates fail in the preclinical test, and it is estimated that more than 90% of cancer-related therapeutics will fail phase I or II clinical trial evaluation. The failure rate in phase III trials is almost 50%, and the cost of new drug development from discovery through phase III trials is between $0.8 billion and $1.7 billion and can take between eight and ten years.

In addition, many subjects fail to respond even to standard drugs that have been shown to be efficacious. Excessive systemic concentrations can be maintained for many oncology drug candidates in efforts to achieve a desired concentration at a tumor site. Introducing oncology drug candidates directly into tumors in lower doses than maintained in systemic concentrations can be useful.

### SUMMARY

In one aspect, the disclosure provides for a method for evaluating an agent in a solid tissue of a subject, comprising:delivering two or more agents to two or more sites in the solid tissue using an administration device, wherein the administration device comprises two or more needles in an array, wherein a first needle of the two or more needles comprises a first agent of the two or more agents and a second needle of the two or more needles comprises a second agent of the two or more agents, performing mass spectrometry on a sample comprising the two or more sites, and evaluating the two or more agents for efficacy.

In some embodiments, the administration device is configured to deliver the two or more agents at an amount undetectable outside the solid tissue, or at a therapeutically effective amount. In some embodiments, the two or more agents are delivered at or below a systemically detectable concentration. In some embodiments, the delivering is performed in vivo. In some embodiments, the delivering is performed in vitro. In some embodiments, a region of permeation of the first agent in the solid tissue is separate from a region of permeation of the second agent in the solid tissue. In some embodiments, an amount of the two or more agents in the two or more needles is less than 5 microliters per needle. In some embodiments, an amount of the two or more agents in the two or more needles is about 4 microliters per needle. In some embodiments, the two or more agents are injected into the two or more sites. In some embodiments, the administration device is configured for passive delivery of the two or more agents into the solid tissue. In some embodiments, the two or more agents are delivered simultaneously to the solid tissue. In some embodiments, the two or more agents are delivered sequentially to the solid tissue. In some embodiments, the two or more needles comprise at least one diffusion device. In some embodiments, the diffusion device comprises a semi-permeable membrane. In some embodiments, the diffusion device comprises a microdialysis probe. In some embodiments, the two or more needles are charged with the agent by immersion in the two or more agents. In some embodiments, the two or more agents are loaded into the two or more needles using a pump, wherein both ends of the two or more needles are open. In some embodiments, the two or more agents are loaded into distal ends of the two or more needles. In some embodiments, the two or more agents are loaded into the two or more needles wherein at least one end of the two or more needles is closed. In some embodiments, the administration device comprises 6 or more needles. In some embodiments, the administration device comprises 10 or more needles. In some embodiments, the administration device comprises 100 or more needles.

In some embodiments, the delivering comprises delivering a first agent systemically to a subject and a second agent locally to the solid tissue. In some embodiments, the method further comprising delivering a first agent to a site and delivering a second agent to the same site. In some embodiments, the method further comprises repeating the delivering step to deliver the two or more agent to the same two or more sites. In some embodiments, the two or more sites are removed by a method from the group consisting of: laser-capture microdissection, histological sectioning, skin punch biopsy, and contact blotting, or any combination thereof. In some embodiments, the method further comprises removing the solid tissue. In some embodiments, the method further comprises removing the two or more sites. In some embodiments, the removing is performed by a biopsy needle. In some embodiments, the removing is performed by the two or more needles. In some embodiments, the two or more sites are removed by skin punch biopsy. In some embodiments, the removing removes an area comprising the site. In some embodiments, the removing removes a columnar region of the solid tissue. In some embodiments, the columnar region comprises the site. In some embodiments, the removing removes an area adjacent to the site.

In some embodiments, the mass spectrometry is imaging mass spectroscopy. In some embodiments, the mass spectrometry is tandem MS/MS. In some embodiments, the mass spectrometry is Matrix-Assisted Laser Desorption Ionization Mass Spectrometry (MALDI MS). In some embodiments, the solid tissue is irradiated with continuous laser spots. In some embodiments, the continuous laser spots have a diameter in a range from about 10 to about 100 microns. In some embodiments, the continuous laser spots have a diameter of about 25 microns. In some embodiments, the mass spectrometry is selected from the group consisting of: electrospray ionization mass spectrometry, multidimensional protein identification technology, and liquid chromatography mass spectrometry, or any combination thereof. In some embodiments, the first agent and the second agent are the same agent. In some embodiments, at least one of the two or more agents comprises a chemotherapeutic agent. In some embodiments, at least one of the two or more agents comprises a small molecule agent. In some embodiments, at least one of the two or more agents comprises an anti-cancer agent. In some embodiments, at least one of the two or more agents comprises an agent that interferes with RNA activity. In some embodiments, at least one of the two or more agents comprises an antibody-drug conjugate. In some embodiments, at least one of the two or more agents comprises a gene therapy agent. In some embodiments, at least one of the two or more agents comprises a biologic. In some embodiments, at least one of the two or more agents comprises an agent in the Orange Book. In some embodiments, at least one of the two or more agents comprises an agent in a clinical trial. In some embodiments, at least one of the two or more agents comprises an agent in an investigational new drug (IND) trial. In some embodiments, at least one of the two or more agents comprises a pharmacological carrier. In some embodiments, at least one of the two or more agents comprises a control agent. In some embodiments, at least one of the two or more agents comprises a positive control. In some embodiments, at least one of the two or more agents comprises a negative control. In some embodiments, at least one of the two or more agents comprises a position marker. In some embodiments, the position marker is used to orient an image of the solid tissue. In some embodiments, the position marker is used as a reference to determine which of the two or more agents are located in which of the two or more sites. In some embodiments, at least one of the two or more agents comprises an agent selected from the group consisting of: a protein, a peptide, a peptidomimetic, an antibody, a small molecule, a small interfering RNA-encoding polynucleotide, an antisense RNA-encoding polynucleotide, antibody-drug conjugate, and a ribozyme-encoding polynucleotide. In some embodiments, the two or more agents is 10 or more agents.

In some embodiments, the solid tissue is subcutaneous. In some embodiments, the solid tissue comprises a tissue selected from the group consisting of: brain, liver, lung, kidney, prostate, ovary, spleen, lymph node, thyroid, pancreas, heart, skeletal muscle, intestine, larynx, esophagus, and stomach. In some embodiments, the solid tissue comprises a tumor. In some embodiments, the solid tissue comprises a skin-related tumor. In some embodiments, the solid tissue comprises melanoma. In some embodiments, the solid tissue comprises lymphoma. In some embodiments, the solid tissue comprises a tumor of unknown origin. In some embodiments, the tumor is selected from the group consisting of: a primary tumor, an invasive tumor and a metastatic tumor. In some embodiments, the solid tissue comprises at least one cancer cell selected from the group consisting of: a prostate cancer cell, a breast cancer cell, a colon cancer cell, a lung cancer cell, a skin cancer cell, a brain cancer cell, and an ovarian cancer cell. In some embodiments, the solid tissue comprises a cancer selected from the group consisting of adenoma, adenocarcinoma, squamous cell carcinoma, basal cell carcinoma, small cell carcinoma, large cell undifferentiated carcinoma, chondrosarcoma, melanoma, lymphoma, and fibrosarcoma.

In some embodiments, the performing mass spectrometry comprises analyzing an atomic mass window of interest to determine the spatial arrangement of a biomarker within the solid tissue. In some embodiments, the analyzing the atomic mass window of interest comprises graphically depicting a mass of the biomarker along X and Y axes of the two or more sites. In some embodiments, the analyzing the atomic mass window of interest comprises graphically depicting a mass of the biomarker along X and Y axes of the solid tissue. In some embodiments, the method further comprises depicting a mass of the biomarker along a Z axis of the solid tissue. In some embodiments, the method further comprises quantifying an intensity of a signal of one or more mass spectrums of the biomarker. In some embodiments, the intensity correlates to a presence or absence of the biomarker. In some embodiments, the intensity correlates to a concentration of the biomarker. In some embodiments, the method further comprises depicting the intensity on the solid tissue. In some embodiments, the graphically depicting the mass of the biomarker comprises graphically depicting in a three dimensional plot. In some embodiments, the mass spectrometry is performed in situ.

In some embodiments, the evaluating comprises determining rates of diffusion of at least one of the two or more agents. In some embodiments, the evaluating comprises determining dosages for systemic use of at least one of the two or more agents. In some embodiments, the evaluating comprises determining a physiological response. In some embodiments, the physiological response is correlated to an agent responsible for the physiological response. In some embodiments, the evaluating comprises pooling mass spectrums. In some embodiments, the evaluating comprises combining mass spectrums from the two or more sites. In some embodiments, the evaluating comprises detecting a biological modification. In some embodiments, the biological modification comprises a modification to a polypeptide. In some embodiments, the biological modification comprises a modification to a nucleic acid. In some embodiments, the biological modification is selected from the group consisting of: phosphorylation, ubiquitylation, methylation, acetylation, glycosylation, and degradation, or any combination thereof. In some embodiments, the evaluating comprises detecting a concentration of a biomarker. In some embodiments, the evaluating comprises detecting a presence or absence of a biomarker. In some embodiments, the presence or absence of the biomarker indicates a genotype of the solid tissue. In some embodiments, the genotype is used to categorize the solid tissue. In some embodiments, the evaluating comprises mapping a distribution of a biomarker on an image of the solid tissue. In some embodiments, the distribution determines an efficacy of the agent. In some embodiments, the distribution determines a permeation of the agent through the solid tissue. In some embodiments, the evaluating comprises determining a genotype of the solid tissue. In some embodiments, the evaluating is used to categorize the solid tissue. In some embodiments, the evaluating is used to categorize the subject for participation in a clinical trial. In some embodiments, the evaluating comprises detecting a degree of permeation of at least one of the two or more agents through the solid tissue. In some embodiments, the evaluating comprises assessing the activity of at least one of the two or more agents on the solid tissue. In some embodiments, the evaluating comprises comparing an activity the first agent with the second agent on the solid tissue. In some embodiments, the evaluating comprises assessing a toxicity of the two or more agents on the solid tissue. In some embodiments, the evaluating comprises comparing a mass spectrum of the two or more sites with a reference mass spectrum. In some embodiments, a first site of the one or more sites comprises an agent and a second site of the one or more sites comprises a control agent. In some embodiments, the evaluating comprises comparing a mass spectrum of the first site with a mass spectrum of the second site. In some embodiments, the evaluating comprises identifying at least one biomarker for the agent by comparing a mass spectrum of the first site with a mass spectrum of the second site. In some embodiments, the evaluating comprises detecting a change in the levels of at least one biomarker for the agent by comparing a mass spectrum of the first site with a mass spectrum of the second site. In some embodiments, the identifying comprises searching a database to identify the at least one biomarker. In some embodiments, the biomarker indicates the genotype of the solid tissue. In some embodiments, the method further comprises administering a treatment regimen specific to the genotype.

In one aspect the disclosure provides for a computer readable medium comprising code that, upon execution by one or more processors, implements the method of evaluating an agent in a solid tissue of a subject, comprising: delivering two or more agents to two or more sites in the solid tissue using an administration device, wherein the administration device comprises two or more needles in an array, wherein a first needle of the two or more needles comprises a first agent of the two or more agents and a second needle of the two or more needles comprises a second agent of the two or more agents, performing mass spectrometry on a sample comprising the two or more sites, and evaluating the two or more agents for efficacy.

In one aspect the disclosure provides for a system for implementing the method of evaluating an agent in a solid tissue of a subject, comprising: delivering two or more agents to two or more sites in the solid tissue using an administration device, wherein the administration device comprises two or more needles in an array, wherein a first needle of the two or more needles comprises a first agent of the two or more agents and a second needle of the two or more needles comprises a second agent of the two or more agents, performing mass spectrometry on a sample comprising the two or more sites, and evaluating the two or more agents for efficacy, comprising: a storage memory for storing a dataset associated with a sample, a processor communicatively coupled to the storage memory, and a dataset. In some embodiments, the data relating to the classification is transmitted over a network.

In one aspect the disclosure provides for a method for evaluating an agent in a solid tissue of a subject, comprising: delivering a combination of two agents to a first site in the solid tissue using an administration device, wherein the administration device comprises two or more needles, wherein a first needle of the two or more needles comprises a first agent and a second needle of the two or more needles comprises a second agent, delivering one of the two agents to a second site in the solid tissue, and comparing the first site with the second site, wherein the comparing comprises comparing the agents for a physiological effect.

In one aspect the disclosure provides for a method for evaluating an agent in a solid tissue of a subject, comprising delivering a first agent to a site in the solid tissue using an administration device, wherein the administration device comprises two or more needles in an array, delivering a second agent systemically to a subject, evaluating the first agent for a physiological effect using mass spectrometry.

In one aspect the disclosure provides for a method for evaluating an agent in a solid tissue of a subject, comprising: delivering two or more agents to two or more sites in the solid tissue using an administration device, wherein the administration device comprises two or more microdialysis probes, wherein a first microdialysis probe of the two or more microdialysis probes comprises a first agent of the two or more agents and a second microdialysis probe of the two or more microdialysis probes comprises a second agent of the two or more agents, performing mass spectrometry on a sample comprising the two or more sites, and evaluating the two or more agents for efficacy.

In some embodiments, the microdialysis probes are delivered to the same site. In some embodiments, the microdialysis probes continually release the two or more agents. In some embodiments, the microdialysis probes sequentially release the two or more agents. In some embodiments, the evaluating evaluates a combination of the two or more agents. In some embodiments, the first agent is delivered first and the second agent is delivered second. In some embodiments, the first agent synchronizes the cells in the cell cycle. In some embodiments, the second agent induces a change in the cell cycle. In some embodiments, a first agent of the two or more agents is delivered systemically to a subject and a second agent of the two or more agents is delivered locally to the solid tissue. In some embodiments, the two or more agents are loaded into the two or more microdialysis probes using a pump, wherein both ends of the two or more microdialysis probes are open. In some embodiments, the two or more agents are loaded into distal ends of the two or more microdialysis probes. In some embodiments, the two or more agents are loaded into the two or more microdialysis probe wherein at least one end of the two or more microdialysis probes is closed.

In some embodiments, the administration device is configured to deliver the two or more agents at an amount undetectable outside the solid tissue, or at a therapeutically effective amount. In some embodiments, the two or more agents are delivered at or below a systemically detectable concentration. In some embodiments, the delivering is performed in vivo. In some embodiments, the delivering is performed in vitro. In some embodiments, a region of permeation of the first agent in the solid tissue is separate from a region of permeation of the second agent in the solid tissue. In some embodiments, an amount of the two or more agents in the two or more needles is less than 5 microliters per needle. In some embodiments, the two or more agents are injected into the two or more sites. In some embodiments, the administration device is configured for passive delivery of the two or more agents into the solid tissue. In some embodiments, the two or more agents are delivered simultaneously to the solid tissue. In some embodiments, the two or more agents are delivered sequentially to the solid tissue.

In some embodiments, the delivering comprises delivering a first agent systemically to a subject and a second agent locally to the solid tissue. In some embodiments, the method further comprising delivering a first agent to a site and delivering a second agent to the same site. In some embodiments, the method further comprises repeating the delivering step to deliver the two or more agent to the same two or more sites. In some embodiments, the two or more sites are removed by a method from the group consisting of: laser-capture microdissection, histological sectioning, skin punch biopsy, and contact blotting, or any combination thereof. In some embodiments, the method further comprises removing the solid tissue. In some embodiments, the method further comprises removing the two or more sites. In some embodiments, the removing is performed by a biopsy needle. In some embodiments, the removing is performed by the two or more needles. In some embodiments, the two or more sites are removed by skin punch biopsy. In some embodiments, the removing removes an area comprising the site. In some embodiments, the removing removes a columnar region of the solid tissue. In some embodiments, the columnar region comprises the site. In some embodiments, the removing removes an area adjacent to the site.

In some embodiments, the mass spectrometry is imaging mass spectroscopy. In some embodiments, the mass spectrometry is tandem MS/MS. In some embodiments, the mass spectrometry is Matrix-Assisted Laser Desorption Ionization Mass Spectrometry (MALDI MS). In some embodiments, the solid tissue is irradiated with continuous laser spots. In some embodiments, the continuous laser spots have a diameter in a range from about 10 to about 100 microns. In some embodiments, the continuous laser spots have a diameter of about 25 microns. In some embodiments, the mass spectrometry is selected from the group consisting of: electrospray ionization mass spectrometry, multidimensional protein identification technology, and liquid chromatography mass spectrometry, or any combination thereof. In some embodiments, the first agent and the second agent are the same agent. In some embodiments, at least one of the two or more agents comprises a chemotherapeutic agent. In some embodiments, at least one of the two or more agents comprises a small molecule agent. In some embodiments, at least one of the two or more agents comprises an anti-cancer agent. In some embodiments, at least one of the two or more agents comprises an agent that interferes with RNA activity. In some embodiments, at least one of the two or more agents comprises an antibody-drug conjugate. In some embodiments, at least one of the two or more agents comprises a gene therapy agent. In some embodiments, at least one of the two or more agents comprises a biologic. In some embodiments, at least one of the two or more agents comprises an agent in the Orange Book. In some embodiments, at least one of the two or more agents comprises an agent in a clinical trial. In some embodiments, at least one of the two or more agents comprises an agent in an investigational new drug (IND) trial. In some embodiments, at least one of the two or more agents comprises a pharmacological carrier. In some embodiments, at least one of the two or more agents comprises a control agent. In some embodiments, at least one of the two or more agents comprises a positive control. In some embodiments, at least one of the two or more agents comprises a negative control. In some embodiments, at least one of the two or more agents comprises a position marker. In some embodiments, the position marker is used to orient an image of the solid tissue. In some embodiments, the position marker is used as a reference to determine which of the two or more agents are located in which of the two or more sites. In some embodiments, at least one of the two or more agents comprises an agent selected from the group consisting of: a protein, a peptide, a peptidomimetic, an antibody, a small molecule, a small interfering RNA-encoding polynucleotide, an antisense RNA-encoding polynucleotide, antibody-drug conjugate, and a ribozyme-encoding polynucleotide. In some embodiments, the two or more agents is 10 or more agents.

In some embodiments, the solid tissue is subcutaneous. In some embodiments, the solid tissue comprises a tissue selected from the group consisting of: brain, liver, lung, kidney, prostate, ovary, spleen, lymph node, thyroid, pancreas, heart, skeletal muscle, intestine, larynx, esophagus, and stomach. In some embodiments, the solid tissue comprises a tumor. In some embodiments, the solid tissue comprises a skin-related tumor. In some embodiments, the solid tissue comprises melanoma. In some embodiments, the solid tissue comprises lymphoma. In some embodiments, the solid tissue comprises a tumor of unknown origin. In some embodiments, the tumor is selected from the group consisting of: a primary tumor, an invasive tumor and a metastatic tumor. In some embodiments, the solid tissue comprises at least one cancer cell selected from the group consisting of: a prostate cancer cell, a breast cancer cell, a colon cancer cell, a lung cancer cell, a skin cancer cell, a brain cancer cell, and an ovarian cancer cell. In some embodiments, the solid tissue comprises a cancer selected from the group consisting of adenoma, adenocarcinoma, squamous cell carcinoma, basal cell carcinoma, small cell carcinoma, large cell undifferentiated carcinoma, chondrosarcoma, melanoma, lymphoma, and fibrosarcoma.

In some embodiments, the performing mass spectrometry comprises analyzing an atomic mass window of interest to determine the spatial arrangement of a biomarker within the solid tissue. In some embodiments, the analyzing the atomic mass window of interest comprises graphically depicting a mass of the biomarker along X and Y axes of the two or more sites. In some embodiments, the analyzing the atomic mass window of interest comprises graphically depicting a mass of the biomarker along X and Y axes of the solid tissue. In some embodiments, the method further comprises depicting a mass of the biomarker along a Z axis of the solid tissue. In some embodiments, the method further comprises quantifying an intensity of a signal of one or more mass spectrums of the biomarker. In some embodiments, the intensity correlates to a presence or absence of the biomarker. In some embodiments, the intensity correlates to a concentration of the biomarker. In some embodiments, the method further comprises depicting the intensity on the solid tissue. In some embodiments, the graphically depicting the mass of the biomarker comprises graphically depicting in a three dimensional plot. In some embodiments, the mass spectrometry is performed in situ.

In some embodiments, the evaluating comprises determining rates of diffusion of at least one of the two or more agents. In some embodiments, the evaluating comprises determining dosages for systemic use of at least one of the two or more agents. In some embodiments, the evaluating comprises determining a physiological response. In some embodiments, the physiological response is correlated to an agent responsible for the physiological response. In some embodiments, the evaluating comprises pooling mass spectrums. In some embodiments, the evaluating comprises combining mass spectrums from the two or more sites. In some embodiments, the evaluating comprises detecting a biological modification. In some embodiments, the biological modification comprises a modification to a polypeptide. In some embodiments, the biological modification comprises a modification to a nucleic acid. In some embodiments, the biological modification is selected from the group consisting of: phosphorylation, ubiquitylation, methylation, acetylation, glycosylation, and degradation, or any combination thereof. In some embodiments, the evaluating comprises detecting a concentration of a biomarker. In some embodiments, the evaluating comprises detecting a presence or absence of a biomarker. In some embodiments, the presence or absence of the biomarker indicates a genotype of the solid tissue. In some embodiments, the genotype is used to categorize the solid tissue. In some embodiments, the evaluating comprises mapping a distribution of a biomarker on an image of the solid tissue. In some embodiments, the distribution determines an efficacy of the agent. In some embodiments, the distribution determines a permeation of the agent through the solid tissue. In some embodiments, the evaluating comprises determining a genotype of the solid tissue. In some embodiments, the evaluating is used to categorize the solid tissue. In some embodiments, the evaluating is used to categorize the subject for participation in a clinical trial. In some embodiments, the evaluating comprises detecting a degree of permeation of at least one of the two or more agents through the solid tissue. In some embodiments, the evaluating comprises assessing the activity of at least one of the two or more agents on the solid tissue. In some embodiments, the evaluating comprises comparing an activity the first agent with the second agent on the solid tissue. In some embodiments, the evaluating comprises assessing a toxicity of the two or more agents on the solid tissue. In some embodiments, the evaluating comprises comparing a mass spectrum of the two or more sites with a reference mass spectrum. In some embodiments, a first site of the one or more sites comprises an agent and a second site of the one or more sites comprises a control agent. In some embodiments, the evaluating comprises comparing a mass spectrum of the first site with a mass spectrum of the second site. In some embodiments, the evaluating comprises identifying at least one biomarker for the agent by comparing a mass spectrum of the first site with a mass spectrum of the second site. In some embodiments, the evaluating comprises detecting a change in the levels of at least one biomarker for the agent by comparing a mass spectrum of the first site with a mass spectrum of the second site. In some embodiments, the identifying comprises searching a database to identify the at least one biomarker. In some embodiments, the biomarker indicates the genotype of the solid tissue. In some embodiments, the method further comprises administering a treatment regimen specific to the genotype.

In one aspect the disclosure provides for a computer readable medium comprising code that, upon execution by one or more processors, implements the method evaluating an agent in a solid tissue of a subject, comprising: delivering two or more agents to two or more sites in the solid tissue using an administration device, wherein the administration device comprises two or more microdialysis probes, wherein a first microdialysis probe of the two or more microdialysis probes comprises a first agent of the two or more agents and a second microdialysis probe of the two or more microdialysis probes comprises a second agent of the two or more agents, performing mass spectrometry on a sample comprising the two or more sites, and evaluating the two or more agents for efficacy.

In one aspect the disclosure provides for a system for implementing the method evaluating an agent in a solid tissue of a subject, comprising: delivering two or more agents to two or more sites in the solid tissue using an administration device, wherein the administration device comprises two or more microdialysis probes, wherein a first microdialysis probe of the two or more microdialysis probes comprises a first agent of the two or more agents and a second microdialysis probe of the two or more microdialysis probes comprises a second agent of the two or more agents, performing mass spectrometry on a sample comprising the two or more sites, and evaluating the two or more agents for efficacy, comprising: a storage memory for storing a dataset associated with a sample, a processor communicatively coupled to the storage memory, and a dataset. In some embodiments, the data relating to the classification is transmitted over a network.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts an exemplary embodiment of the methods of the disclosure.

FIG. 2 depicts a system for software facilitation.

FIG. 3 depicts an exemplary injection device of the disclosure.

FIG. 4 depicts an exemplary needle assembly device of the disclosure.

### DETAILED DESCRIPTION

### Definitions

As used herein the term “about” can refer to ±10% and includes ±1% and ±0.1%.

As used herein the term “agent” or “agents” can refer to any substance capable of flowing through a needle or diffusing through a microdialysis probe. Agents can refer to liquids, gases, colloids, and suspended solids. Agents may be dissolved or suspended in an aqueous solution as a mixture or colloid that may be delivered to a target tissue. Agents can be used interchangably with “therapeutic agents,” “visualizing agent,” and “candidate agents.” An agent can be any substance capable of being delivered through a needle, including liquids, gases, colloids, suspended solids, etc. In some instances, “agents” can refer to two agents which can be the same agent at different concentrations. For example, prednisone at a concentration of 1 mM and at 10 mM can be two agents.

As used herein, “altered physiologic state” can refer to any detectable parameter that directly relates to a condition, process, pathway, dynamic structure, state or other activity in a solid tissue (and in some embodiments in a solid tumor) including in a region or a biological sample that permits detection of an altered (e.g., measurably changed in a statistically significant manner relative to an appropriate control) structure or function in a biological sample from a subject or biological source. An altered physiologic state can include, for example, altered cellular or biochemical activity (e.g., a cell viability, cell proliferation, apoptosis, cellular resistance to anti-growth signals, cell motility, cellular expression or elaboration of connective tissue-degrading enzymes, and/or cellular recruitment of angiogenesis).

Altered physiologic state can refer to any condition or function where any structure or activity that is directly or indirectly related to a solid tissue function has been changed in a statistically significant manner relative to a control or standard. Altered physiologic state can have its origin in direct or indirect interactions between a solid tissue constituent and an introduced agent, or in structural or functional changes that occur as the result of interactions between intermediates that can be formed as the result of such interactions, including metabolites, catabolites, substrates, precursors, cofactors and the like. Additionally, altered physiologic state can include altered signal transduction, respiratory, metabolic, genetic, biosynthetic or other biochemical or biophysical activity in some or all cells or tissues of a subject or biological source, in some or all cells of a solid tissue, and in some or all cells of a tumor such as a solid tumor in a solid tissue. As non-limiting examples, altered biological signal transduction, cell viability, cell proliferation, apoptosis, cellular resistance to anti-growth signals, cell motility, cellular expression or elaboration of connective tissue-degrading enzymes, cellular recruitment of angiogenesis, or other criteria including induction of apoptotic pathways and formation of atypical chemical and biochemical crosslinked species within a cell, whether by enzymatic or non-enzymatic mechanisms, can all be regarded as indicative of altered physiologic state.

As used herein, the term “biomarker” can refer to proteins, peptides, amino acids, RNA, DNA, nucleic acids, proteoglycans, lipids, small organic molecules, small inorganic molecules, or ions. A biomarker can be any biological molecule. A biomarker can be indicative of an effect of an agent and/or of a pathway or process. Biomarkers can be measured in transcriptional levels as gene expression of both DNA and RNA or in protein levels.

As used herein the term “negative control” can refer to any control agent that may cause no statistically significant alteration of physiological state. A negative control can refer to sham injections, saline, DMSO buffer control, inactive enantiomers, scrambled peptides or nucleotides, and delivery vehicles.

As used herein, the term “position marker” can refer to compositions that spatially define location in a solid tissue, tumor, or cell. Position markers can refer to nanoparticles, nanostructures, dyes, fluorescent compounds, enzyme substrates, specific oligonucleotide probes, reporter genes, metal or plastic clips, fluorescent quantum dots, India ink, metal or plastic beads, dyes, stains, tumor paint, and the like. Markers can include any subsequently locatable source of detectable signal, which can be visible, optical, colorimetric, originate from a dye, enzymatic, originate from a GCMS tag, originate from a radiological (including radioactive radiolabel and radio-opaque), fluorescent or other any other detectable signal. Positional markers can also refer to microdialysis tubes, or colored microdialysis probe or needles.

As used herein the term “pre-determined period of time” or “selected period of time” can refer to any time within a range of 1 minute to 2 years. In some embodiments, the pre-determined period of time or selected period of time is at most about 0.1, 0.2, 0.5, 1, 2, 3, 4, 5, 6, 7, 8, 9, 10, 11, 12, 15, 18, 24, 36, 48, 72, 96, 120, 144, 168, or 192 hours. In some embodiments, the pre-determined period of time or selected period of time is at least about 0.1, 0.2, 0.5, 1, 2, 3, 4, 5, 6, 7, 8, 9, 10, 11, 12, 15, 18, 24, 36, 48, 72, 96, 120, 144, 168, or 192 hours. In some other embodiments, the pre-determined period of time or selected period of time is in a range of about 24-72 hours.

As used herein, the term “small molecule agent” can refer to an agent with a molecule weight less than about 1000 daltons, less than about 800 daltons, or less than about 500 daltons. A small molecule agent can be an anti-cancer agent.

As used herein, the term “solid tissue” may refer to tissue of the brain, liver, lung, kidney, prostate, ovary, spleen, lymph node, thyroid, pancreas, heart, skeletal muscle, intestine, larynx, esophagus, stomach, and the like. Solid tissue may also refer to a tumor, which may be benign or malignant, invasive, metastatic, or primary. Solid tissue may refer to a cancer selected from adenoma, adenocarcinoma, squamous cell carcinoma, basal cell carcinoma, small cell carcinoma, large cell undifferentiated carcinoma, chondrosarcoma and fibrosarcoma. In some embodiments, the solid tissue may refer to at least one cancer cell selected from the group consisting of a prostate cancer cell, a breast cancer cell, a colon cancer cell, a lung cancer cell, a brain cancer cell, and an ovarian cancer cell.

As used herein the term “synergistic activity or toxicity” can refer to coordinated activity or toxicity of two or more agents such that the combined action is greater than the sum of each agent acting separately. The coordinated activity or toxicity may be at least about 10%, 20%, 30%, 40%, 50%, 60%, 70%, 80%, 90%, 100%, 200%, 300%, 400%, 500% or even higher than the sum of each agent acting separately.

As used herein the term “therapeutically effective concentrations” can refer to the concentration of an agent in a solid tissue when a desirable pharmacological effect is observed. For example, an anti-cancer agent can enter systemic circulation after an absorption process. In some instances, systemic circulation can facilitate accumulation of the agent in a solid tissue. The concentration of the agent in the solid tissue and in systemic circulation may be the same or different when a desirable pharmacological effect is observed.

As used herein the term “transgenic animal” can refer to a non-human animal in which one or more of the cells of the animal includes a nucleic acid that is not endogenous (i.e., heterologous). The nucleic acid can be present as an extrachromosomal element in a portion a cell or stably integrated into germ line DNA.

General Overview

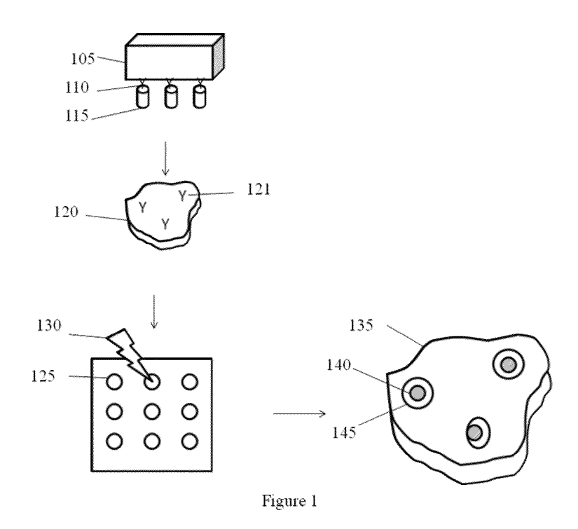

The present disclosure provides methods for multiplexed delivery and evaluation of agents using mass spectrometry. FIG. 1 shows and exemplary method according to the disclosure. A needle array device for inserting agents 105 can comprise a plurality of needles 115. A needle 115 can comprise a microdialysis probe 110 which can comprise the agent. The needles 115 can be inserted in a solid tissue 120, whereupon agents and/or the microdialysis probe 110 can be inserted into a plurality of sites 121 within the solid tissue 120. In some instances, where insertion comprises injection of a microdialysis probe 110, withdrawal of the needles 115 inserts and leaves the microdialysis probes 110 in the solid tissue 120. The plurality of injection sites 121 in the solid tissue can be collected and made suitable for being placed on a mass spectrometry array plate 125 which can be suitable for MALDI-type mass spectrometry. The plate can be irradiated with a laser pulse 130 which can allow the agent to be detected by the mass spectrometer. The abundance and/or distribution of the agent, and/or physiologic effect caused by the agent, can be graphically mapped on an image of the tumor 135. In a non-limiting example, a section of the solid tissue may display an agent profile that comprises a central region of a high concentration of agent and/or physiologic effect 140 surrounded by a region represents a lower concentration of agent and/or physiologic effect 145.

Devices

Devices and methods for delivery using a needle array are described in PCT/US2008/073212, filed Aug. 14, 2008.

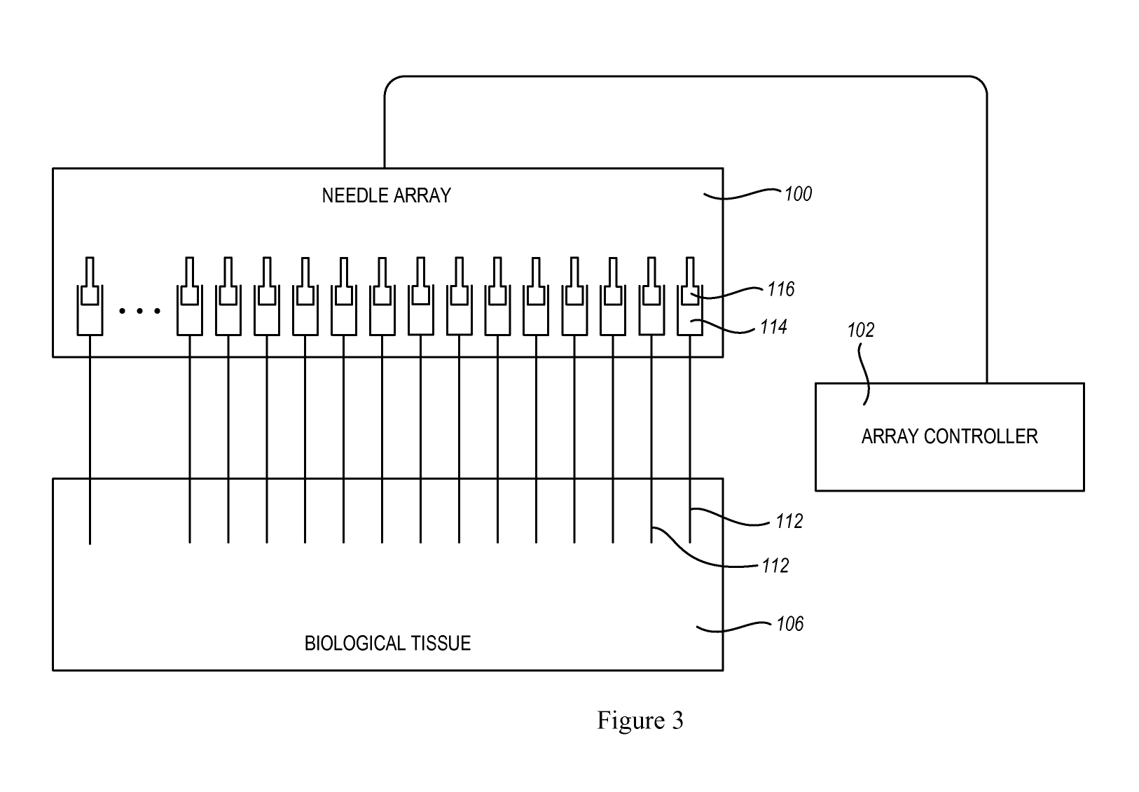

Referring to FIG. 3, a needle array assembly 100 is shown, including a plurality of needles 112, a plurality of reservoirs containing porous tubes 114, a plurality of delivery actuators such as, in the present example, plungers 116, and a controller 102. One or more of the plurality of needles 112 can be fixed in position relative to the others of the plurality of needles, and the plungers can be likewise operatively coupled so as to be fixed in position and simultaneously actuable. One or more of the plurality of needles 112 can be in fluid communication with a respective one of the plurality of reservoirs 114, and one or more of the plurality of plungers includes a first end positioned in a respective one of the plurality of reservoirs 114. The controller 102 can be operatively coupled to second ends of each of the plurality of plungers 116. The controller can be configured to control actuation of the plungers within the reservoir with respect to speed, distance, and direction of movement.

One or more of the porous tubes within a reservoir 114 can be charged with a different agent, or some or all of the porous tubes can be charged with a common agent. Movement of the plurality of plungers 116 in a second direction can create a positive pressure, or overpressure, in the respective reservoirs 114, forcing the contents of the reservoirs out via the respective needles 112.

In this configuration, a relatively small amount of a plurality of therapeutic agents can be simultaneously inserted directly to a region of solid tissue 106 for evaluation and analysis. Following insertion, an agent within a needle can be released to the surrounding tissue by passive diffusion. In some embodiments, the amount of a therapeutic agent delivered to the tissue can be less than 1 microliter per needle. In some embodiments, the amount of an agent delivered to the tissue can be more than 1 microliter per needle, but less than 10 microliters. In some embodiments, the amount of agent delivered to the tissue can be less than about 1, 10, 50, 100, 500, 1000 microliters. In some embodiments, the amount of agent delivered to the tissue can be more than about 1, 10, 50, 100, 500, 1000 microliters. In some embodiments, the amount of agent delivered can be about 4 microliters. The evaluation of the tissue 106 and the efficacy of the different therapeutic agents delivered thereto can be used, for example, to screen potential therapeutic agents for subsequent clinical trials (e.g., perform exploratory studies (e.g., investigational new drug studies)), microdose studies, and/or to make subject-specific treatment decisions based on the relative efficacy of the therapeutic agents in the tissue 106.

Any number of needles can be used in the needle array device. The needle array device can comprise at least 1, 2, 3, 4, 5, 6, 7, 8, 9, 10, 11, 12, 13, 14, 15 16, 17, 18, 19, 20, 30, 40, 50, 60, 70, 80, 90 or more needles. The needle array device can comprise at most 1, 2, 3, 4, 5, 6, 7, 8, 9, 10, 11, 12, 13, 14, 15 16, 17, 18, 19, 20, 30, 40, 50, 60, 70, 80, 90 or more needles. In some instances, a needle array device can comprise more than 1000 needles. In some embodiments, the device can comprise 6 needles. In some embodiments, the device can comprise 10 needles. Needles can be configured for delivery by intramuscular injection. Needles can be hypodermic needles. Hypodermic needles can be thin-walled needles. Hypodermic needles can be regular-wall needles. Hypodermic needles can be 7, 8, 10, 11, 12, 13, 14, 15, 16, 17, 18, 19, 20, 21, 22, 22s, 23, 24, 25, 26, 26s, 27, 28, 29, 30, 31, 32, 33, 34 gauge. Needles can be biopsy needles. Needles can remove a portion of the solid tissue upon removal of the needle from the solid tissue.

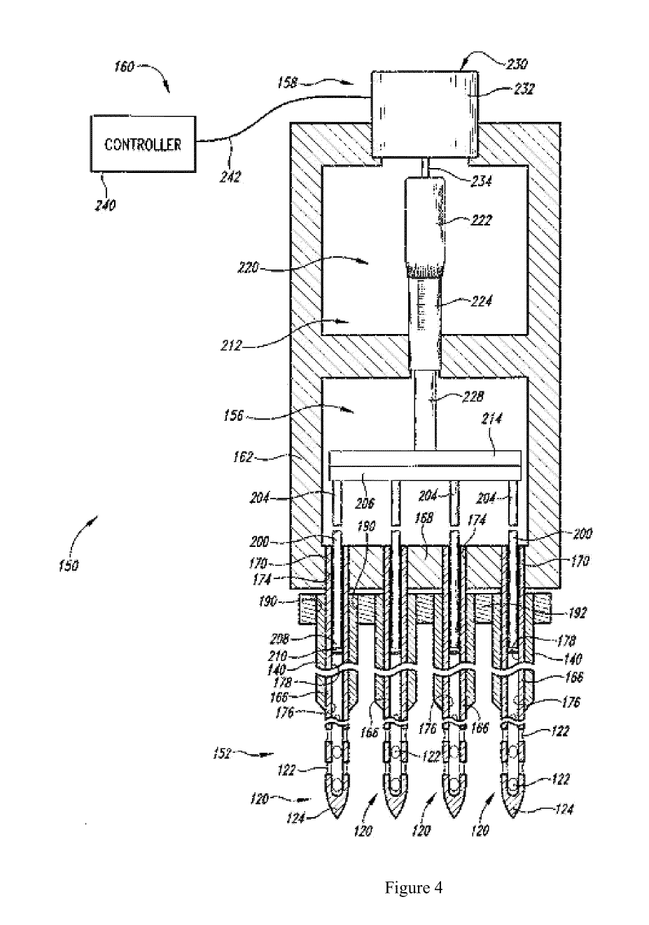

In some instances a needle array can be part of a delivery assembly. FIG. 4 depicts an exemplary diagrammatic view of a delivery assembly 150. The delivery assembly 150 can comprise a needle array 152, an inserter assembly 154, an actuator assembly 156, a driver assembly 158, a control assembly 160, and a frame 162. The frame 162 provides a substantially rigid structure to which other elements of the assembly 150 are coupled.

The needle array 152 can comprise a plurality of needle cylinders 166 and a needle block 168. The needle block 168 can be integral with the frame 162. A needle cylinder 166 can be coupled, at a first end 170, in a respective needle aperture 174 extending in the needle block 168, and can comprise a lumen 176, comprising a nominal diameter extending substantially the entire length of the needle cylinder 166. A needle cylinder 166 can comprise a reservoir 178 in a region toward the first end 170, a needle 120 in a region toward a second end 180, and a tip-end 124 at the second end 180 of the needle cylinder 166. The tip-end 124 can be tapered to a point.

A delivery needle 120 can comprise a plurality of ports 122 distributed along its length. The length of a needle cylinder 166 and of the respective needles 120 can vary. In some instances, a needle cylinder 166 can be longer than 15 centimeters. In some instances, a needle cylinder is at least about 1, 2, 3, 4, 5, 6, 7, 8, 9, 10, 15, 20, 25, 30, 35, 40 or more centimeters long. In some instances, a needle cylinder is at most about 1, 2, 3, 4, 5, 6, 7, 8, 9, 10, 15, 20, 25, 30, 35, 40 or more centimeters long. A delivery needle 120, defined by the portion of the respective needle cylinder 166 along which the ports 122 are spaced, can be at least 1, 2, 3, 4, 5 6, 7, 8, 9, 10, 15, 20 or more centimeters long. A delivery needle can be at most 1, 2, 3, 4, 5 6, 7, 8, 9, 10, 15, 20 or more centimeters long.

The inserter assembly 154 can comprise a plurality of inserter needles 140 coupled to an inserter block 192 in respective inserter apertures 190 extending therein in a configuration that corresponds to the arrangement of the needle cylinders 166 in the needle block 168, such that each of the plurality of needle cylinders 166 can be positioned within a respective one of the plurality of inserter needles 140. The inserter assembly 154 can be axially slidable over the needle cylinders 166 between a first position, in which only the tip-ends 124 of each of the needle cylinders 166 extend from respective ones of the plurality of inserter needles 140, to a second position, in which the second ends 180 of each of the needle cylinders 166 extends from the respective inserter needle 140 a distance sufficient to clear all of the ports 122 of the respective delivery needle 120.

A spacer can be provided which can be configured to be positioned between the inserter block 192 and the needle block 168, sized such that when the inserter block and the needle block are both engaged with the spacer, the inserter block is maintained in the first position. Removal of the spacer may permit movement of the inserter block 192 and the needle block 168 relative to each other, to permit placement of the inserter block into the second position, relative to the needle block.

The actuator assembly 156 can comprise a plurality of plungers 200 coupled at respective first ends 204 to a plunger block 206 in a configuration that corresponds to the arrangement of the needle cylinders 166 and the inserter needles 140 such that a second end 208 of each of the plurality of plungers 200 can be positioned within the reservoir 178 of a respective one of the plurality of the needle cylinders 166. An O-ring 210 can be provided at the second end 208 of each of the plurality of plungers 200 to sealingly engage the wall of the respective lumen 176. The actuator assembly 156 can also comprise an actuator 212 coupled to an actuator block 214, which in turn can be rigidly coupled to the plunger block 206. The actuator 212 can comprise a micrometer device 220 having a thimble 222, a barrel 224, and a spindle 228. The barrel 224 can be rigidly coupled to the frame 162 while the spindle 228 can be rotatably coupled to the actuator block 568 so as to control translational movement of the actuator block relative to the frame 162. The micrometer device 568 can be calibrated in 0.01 mm increments, with a spindle travel of about 0.5 mm per rotation of the thimble 222 and a maximum stroke of 15 mm. Thus, a complete rotation of the thimble can move each of the plurality of plungers 0.5 mm within the lumen 178 of the respective needle cylinder 166 and displaces about 0.0001 cm³ of volume. The dispensing capacity of a needle 120 can be at most about 1, 2, 3, 4, 5, 6, 7, 8, 9, 10 or more microliters. The dispensing capacity of a needles 120 can be at least about 1, 2, 3, 4, 5, 6, 7, 8, 9, 10 or more microliters. In some instances, the dispensing capacity of a needle 120 is about 4 microliters.

The driver assembly 158 can comprise a stepper motor 230. A stepper motor 230 can comprise a motor casing 232, and a motor shaft 234 coupled to a rotor of the motor 230. The motor casing 232 can be rigidly coupled to the frame 162, and the motor shaft 234 can be slidably coupled to the thimble 222 of the micrometer device 568 while being rotationally locked therewith, such as via a spline coupling, for example. Rotational force from the motor shaft 234 can be transmitted to the thimble 222, while axial movement of the thimble may not be limited by the motor shaft. The stepper motor 230 can be configured to divide each rotation into 125 steps. For example, an incremental rotational step of the motor 230 can rotate the thimble about 3°, displacing a volume of about 0.8 picoliter per reservoir 178.

The controller assembly 160 can also comprise a controller 240 and a control cable 242 that can extend from the controller to the stepper motor 230. Signals for controlling direction, speed, and degree of rotation of the motor shaft 234 can be transmitted from the controller 240 to the stepper motor 230 via the control cable 242. The controller can be programmable. A user can program the controller to control a speed of delivery of a fluid from the delivery needles 120 by selecting the speed of rotation, and/or a volume of fluid delivered by selecting the number of partial and complete rotations of the rotor. The controller can be manually operated, such that a user can control a rate and/or direction of rotation of the motor 230 in real time. In some instances, the driver and controller assemblies may be omitted, and a user can control fluid delivery by manually rotating the thimble 222 of the actuator assembly 212.

Charging the reservoirs 178 can be accomplished in a number of ways. For example, a charging vessel can be provided that includes a plurality of cups or compartments in an arrangement that corresponds to the arrangement of the needle cylinders 166. The user can place a selected fluidic agent or combination of agents in each of the cups. The delivery assembly 150 can be positioned with the needle cylinders pointing downward, and the spindle 228 of the actuator 212 fully extended. The frame 162 can be lowered until the needles 120 are fully immersed in the fluids in the respective cups. The motor 230 can be controlled to rotate in the reverse direction, drawing the spindle 228 inward and pulling the plungers 200 upward. This may create a negative pressure in the reservoirs 178 relative to ambient, and draw the fluids into the needle cylinders 166 via the needle ports 122. When the reservoirs are sufficiently charged, rotation of the rotor can be halted and the needle array 152 can be withdrawn from the charging vessel. In some instances, the device may include a back pressure sensing device to deliver a consistent flow rate.

Delivery of the agents in the needle can be performed in a few seconds, or it can be extended over minutes or hours under a relatively low overpressure to promote complete absorption of the fluid into the surrounding tissue. The stepper motor 230 can be controlled to rotate the rotor fast enough to depress the plungers 200 the full length of the needle in less than one second, or slow enough that a single rotation can take many hours. The plungers can be depressed the length of the needle in at least 1 second, 2 seconds, 3 seconds, 4 seconds, 5 seconds, 6 seconds, 7 seconds, 8 seconds, 9 seconds, 10 seconds, 30 seconds, 1 minute, 2 minutes, 3 minutes, 4 minutes, 5 minutes, 6 minutes, 7 minutes, 8 minutes, 9 minutes, 10 minutes, 30 minutes, 1 hour, 2 hours, 3 hours, 4 hours, 5 hours, 6 hours, 7 or more hours. The plungers can be depressed the length of the needle in at most 1 second, 2 seconds, 3 seconds, 4 seconds, 5 seconds, 6 seconds, 7 seconds, 8 seconds, 9 seconds, 10 seconds, 30 seconds, 1 minute, 2 minutes, 3 minutes, 4 minutes, 5 minutes, 6 minutes, 7 minutes, 8 minutes, 9 minutes, 10 minutes, 30 minutes, 1 hour, 2 hours, 3 hours, 4 hours, 5 hours, 6 hours, 7 or more hours.

The length of each of the plurality of needle cylinders and of the respective needles can vary. A needle cylinder can be at least about 1, 2, 3, 4, 5, 6, 7, 8, 9, 10 or more centimeters long. A needle cylinder can be at most about 1, 2, 3, 4, 5, 6, 7, 8, 9, 10 or more centimeters long. A needle cylinder can be 15 centimeters long.

The delivery actuators can be any suitable actuator can be used to control an amount of therapeutic agent delivered from the reservoirs into the needle. For example, fluid pressure such as by compressed air or pressurized liquid can be used to control an amount of therapeutic agent delivered to a region of biological tissue via the porous tubes and needles.

In some instances, agents delivered to a solid tissue can permeate through the solid tissue. Permeation (e.g., diffusion) can occur at different rates depending on the type of agent used. For example, an agent comprising a hydrogel may permeate the solid tissue at a different rate and/or in a different spatial orientation than an agent lacking a hydrogel. In some instances, the region of permeation of a first agent may not overlap with a region of permeation of a second agent. In some instances, a region of permeation of a first injection site comprising a first agent may not overlap with a region of permeation of a second injection site comprising a second agent.

Porous Tubes

In some embodiments, the present method provides for the administration of an agent to a tissue through the use of one or more porous tubes.

A needle array device can comprise a needle, an actuator, a reservoir and/or a plunger, or any combination thereof. A plurality of needles can be attached to a plurality of actuators. The plurality of actuators can be coupled to a plurality of porous tubes or bundles of porous tubes within a plurality of reservoirs, such that depressing the plunger causes ejection of the porous tubes, or injection of the porous tubes into a tissue. In some embodiments, the plungers can be operatively coupled together at respective second ends so as to be simultaneously depressable. A plunger driver can be configured to depress all of the plurality of plungers at a selectively variable rate. Each of the plurality of actuators can comprise one of a plurality of fluid transmission lines having first and second ends, a first end of each of the plurality of fluid transmission lines being coupled to a respective one of the plurality of reservoirs. The device can comprise a fluid pressure source, and each of the plurality of actuators can comprise a fluid coupling between the fluid pressure source and a respective one of the plurality of reservoirs. The fluid pressure source can comprise at least one of a compressor, a vacuum accumulator, a peristaltic pump, a master cylinder, a microfluidic pump, and a valve.

An agent can be loaded into needles, porous tubes, and/or microdialysis probes of the injection device using a peristaltic pump. In some instances, both ends of the needle are open while loading. This configuration may be useful for preclinical studies. In some instances, loading occurs at the distal end of the needle (e.g., through the end of the needle). This configuration may be useful for clinical studies. In some instances, the agents in the needle are removed of bubbles. In some instances, bubble removal occurs while the agent is loaded into the needle.

One or more of the plurality of needles can comprise a plurality of ports distributed along its length. An agent within a porous tube may come into diffusional communication with a solid tissue via these ports, allowing delivery of the agent to the tissue for the duration of needle insertion. The device may be useful for controlling the duration of passive delivery. In some cases, one end of a porous tube may be open, and configured such that application of pressure to this end can determine the rate of delivery of agent from the porous tube.

The agent can contact the tissue by diffusion through pores of the porous tubes. Porous tubes used in the devices and methods of the present application may be hollow, or may uniformly comprise porous material. The porous tubes can be suitable for containing, storing, administering, delivering, and transporting contents. The contents can be a pharmaceutical composition comprising one or more agents. The therapeutic agents within a single hollow and/or porous tube can be the same or can be a mixture of different types of therapeutic agents. Within a plurality of hollow and/or porous tubes, each tube can contain the same therapeutic agents as another tube, or different therapeutic agents as another tube. In some embodiments, every hollow and/or porous tube contains therapeutic agents that are unique from the therapeutic agents contained in every other tube of the plurality of tubes.

The hollow and/or porous tubes can be connected to a frame that holds the tubes and facilitates drug delivery. The hollow and/or porous tubes can be detachable from the frame. The number and spatial orientation of hollow and/or porous tubes connected to the frame can be varied based on the drug-delivery needs of a subject.

A hollow and/or porous tube can be made of a tube material. The tube material can be suitable for containing, storing, administering, delivering, diffusing, and transporting an agent. The contents can be a pharmaceutical composition comprising one or more therapeutic agents. The tube material can be essentially inert to acid. The tube material can be essentially inert to base. The tube material can be essentially inert to acid and base. The tube material can be insoluble in water. The tube material can be insoluble in organic solvents. The tube material can be essentially insoluble in organic solvents. The tube material can be insoluble in non-halogenated organic solvents. The tube material can be essentially insoluble in non-halogenated organic solvents. The tube material can be biocompatible. The tube material can be essentially physiologically-inactive, and may not trigger physiological events. The tube material may not cause inflammation, immune response, infection, or any other sort of rejection within a solid tissue. The tube material can be biodegradable. The tube material can decompose over time within a solid tissue. The tube material can be thermostable, and the tubes can be sterilized in an autoclave prior to use on/in a subject.

The tube material can be suitable for being shaped into a tube, but also suitable for retaining the tube shape upon deposition into solid tissue. The tube material can be suitable for being broken, cut, sliced, disjoined, or separated in a clean way, and can be broken, cut, sliced, disjoined, or separated after deposition into a solid tissue. In some embodiments, the tube material can be scissile.

The tube material can be polymeric. The tube material can be co-polymeric. The tube material can be a cross-linked polymer or co-polymer. Non-limiting examples of tube materials include polysulfone, polyamine, polyamide, polycarbonate, polycarbamate, polyurethane, polyester, polyether, polyolefin, polyaromatic materials. In some embodiments, the tube material can be polysulfone.

The preparation of hollow tubes from polymers can be achieved by various routes. These can be referred to as wet, dry or melt-forming processes. Melt-forming can involve heating a polymer above its melting point and extruding it through an orifice which can be designed to form a hollow tube. Once extruded, the melt can be cooled via a quench which allows the polymer to solidify into a fine tube. In the dry-forming process, a solution of the polymer can be extruded through a desired orifice and can be fed into a heated column which allows for evaporation of the solvent and subsequent formation of a tube. In a wet-membrane forming process, a solution of the polymer can be extruded though an orifice and quenched in a non-solvent for the polymer resulting in coagulation of the polymer to a tube. Of the above mentioned forming processes, wet-membrane forming can allow one to easily produce hollow porous tubes. The particular forming process used will be dependent upon the polymer used and type of hollow tube desired.

In some instances, the tube material can be coated with a substance that can prevent wound healing and/or clot formation. The coating can have antithrombogenic properties such as heparin. The substance can be selected from the group consisting of: a vitamin K antagonist (i.e. coumarins), heparin and heparin derivatives, direct factor Xa inhibitors, direct thrombin inhibitors, antithrombin protein therapeutics, food or herbal supplements, or any combination thereof. Some non limiting examples can include: heparin, anti-thrombin III, fibrin, anti-thromboplastin, heparan sulphate, Protein C, Protein S, Warfarin (Coumadin). Some non limiting examples of Coumarins can include Acenocoumatrol phenprocoumon, Atromentin, Brodifacoum. Heparin and heparin derivatives can include low molecular weight heparin, Vacutainer brand heparin, and heparin made from pig intestines. Inhibitors of factor Xa can be direct or indirect. Some nonlimiting examples of inhibitors of factor Xa can include: Fondaparinux, Idraparinux, rivaroxaban, or apixaban. Antithrombin protein therapeutics can include antithrombin protein that is purified from human plasma or produced recombinantly. One non-limiting example of a recombinatntly produced antithrombin is Atryn. Direct thrombin inhibitors can be used as an anticoagulant. Some non-limiting examples of direct thrombin inhibitors include: hirudin, lepirudin, bivalirudin, argatroban, dabigatran, and ximelagatran. Other types of anticoagulants can include substances isolated from non-human organisms. Some non-limiting examples include Batroxobin, a toxin from snake venom, and Hemetin, an anticoagulant protease.

In some embodiments, the tube material can comprise a plurality of pores. The contents of the tube can diffuse from the tube into solid tissue via the pores. The rate of diffusion from the porous tube into the solid tissue can be influenced by the pore size, for example, larger pores can result in a higher diffusion rate. In some embodiments, the tube material can be permeable. In some embodiments, a porous tube can be permeable.

The agent can diffuse in the direction of lower chemical potential, i.e., toward the exterior surface of the device. At the exterior surface of the device, equilibrium can be again established. A steady state flux of the effective agent can be established in accordance with Fick's Law of Diffusion. The rate of passage of the drug through the material by diffusion can generally be dependent on the solubility of the drug therein, as well as on the thickness of a porous wall. Selection of porous tube materials may depend on the particular agent to be delivered.

In producing a porous material, the size of the pores can be affected by the solvent strength of a polymer. A rapid decrease in solvent strength can entrap a dispersion of small droplets within the continuous polymer phase. A slow decrease in solvent strength can allow for nucleation sites within the polymer matrix allowing for formation of larger pores. In such cases, the reduction in solvent strength can rapid enough to allow for the structure of the membrane to set.

Another way to change porosity and volume of the porous network in producing a porous polymer can be to change the concentration of the polymer solution. Lower concentrations can promote larger pores and greater pore volume. The concentration of the polymer in a solvent can be at least about 10, 20, 30, 40, 50, 60, 70, 80, 90 or 100% w/v. The concentration of the polymer in a solvent can be at most about 10, 20, 30, 40, 50, 60, 70, 80, 90 or 100% w/v. The concentration of polymer in a solvent can be at most 45% w/v. Another method to achieve porous tubular membranes can be to cause a rapid phase inversion of the polymer solution by cooling.

The average pore size can be at least about 50 nm, 100 nm, 200 nm, 300 nm, 400 nm, 500 nm, 600 nm, 700 nm, 800 nm, 900 nm, 1 μm, 2 μm, 3 μm, 5 μm, 10 μm, 20 μm, 30 μm, 50 μm, 100 μm, or 500 μm. The average pore size can be at most about 50 nm, 100 nm, 200 nm, 300 nm, 400 nm, 500 nm, 600 nm, 700 nm, 800 nm, 900 nm, 1 μm, 2 μm, 3 μm, 5 μm, 10 μm, 20 μm, 30 μm, 50 μm, 100 μm, or 500 μm. All the pores of a single tube can be about the same pore size. In some embodiments, each pore of a single tube has a pore size that is independent of the pore size of all the other pores of the tube. Within a plurality of porous tubes, all pores can have about the same pore size, or each pore can have a size that is independent of the size of all the other pores of the plurality of porous tubes.

Within a single porous tube, the pore sizes can vary by as much as 5%, 10%, 15%, 20%, 25%, 30%, 40%, 50%, 60%, 70%, 75%, 80%, 90%, 100%, 125%, 150%, 200%, 300%, 400%, 500%, 750%, or 1,000% or more. The pore size can influence the rate of diffusion, and the pore size can be modulated to influence the rate of diffusion. A porous tube can be generated having a pre-determined average pore size for the purpose of influencing the rate of diffusion. Different pharmaceutical compositions of therapeutic agents can diffuse from the porous tubes at varying rates, influenced in part by the physical and chemical properties of the pharmaceutical compositions, therapeutic agents, and porous tube materials. Porous tubes with varying average pore sizes can be generated and used experimentally to find a pore size that provides a desired diffusion rate for a specific pharmaceutical composition or therapeutic agent.

In a plurality of tubes, all tubes can have about the same tube length, or each tube can have a tube length that is independent of the tube length of all the other tubes of the plurality of tubes. A tube can have a diameter. In a plurality of tubes, all tubes can have about the same diameter, or each tube can have a diameter that is independent of the diameter of all the other tubes of the plurality of tubes. A tube can have a wall thickness. In a plurality of tubes, all tubes can have about the same wall thickness, or each tube can have a wall thickness that is independent of the wall thickness of all the other tubes of the plurality of tubes.

A porous tube can have a top end and a bottom end. The entire tube can comprise an agent, such as a pharmaceutical composition or therapeutic agents. In some embodiments, the bottom end can comprise the agent, and the top end may not comprise an agent. In some embodiments, the top end can comprise the agent, and the bottom end may not comprise an agent.

The bottom end of a tube can be attached to a device suitable for assisting in the administration of the contents into a solid tissue. The bottom end of a tube can be connected, for example, to a needle, port, catheter, intravenous line, or other apparatus suitable for delivering a pharmaceutical composition into a solid tissue. In some embodiments, the apparatus (e.g., a needle) can be suitable for penetrating a solid tissue.

The top end of a tube can be attached to a device suitable for assisting in the administration of the contents into a solid tissue. The top end of a tube can be connected, for example, to a plunger, pump, piston, or other apparatus suitable for providing a pressure sufficient to deliver a pharmaceutical composition into a solid tissue, or any such device described herein.

The tubes can be loaded, packed, or charged with an agent. The tubes can be loaded immediately prior to use, or can be loaded, stored, and shipped. Either end of a porous tube, or both ends, may be sealed following loading with an agent. In some cases, one or both ends of a porous tube may be sealed prior to loading with an agent by, for example, soaking the tube for an extended period of time in the agent.

The narrow diameter and shape of the tubes provides for convenient loading by capillary action. A tube, or a plurality thereof, can be dipped into a fluid pharmaceutical composition, and the pharmaceutical composition can be drawn into the tubes. In a closed environment, the application of positive pressure to the pharmaceutical composition results in loading a greater amount of the pharmaceutical composition into the tubes; thus, the amount of pharmaceutical formulation in a tube can be controlled easily and reliably.

The porosity of the tubes can provide for convenient loading by soaking the tubes in a bath of a fluid pharmaceutical composition. The pharmaceutical composition can diffuse into the tubes, for example, through the pores or via permeability of the tube material. The amount of pharmaceutical composition that diffuses into the tubes can be influenced, for example, by external pressure, pore size, permeability, tube length, bath depth, bath amount, amount of time spent in the bath, and tube material.

Drug Implants

The present invention provides methods for the administration of an agent to a solid tissue through the use of one or more drug implants. Drug implants can comprise a plurality of agents and can be distributed within a solid tissue using a needle array device of the disclosure (e.g., injection device). A device can allow drug implants to be inserted through the introducer needles into the tumor. A drug implant can comprise a solid rod or stick. A drug implant may comprise a drug agent in solid form. In some cases, the drug implant can comprise an inert biodegradable binder that permits sustained or timed release of the drug within the tissue.

In some embodiments, a drug implant can have a diameter of less than about 0.001, 0.01, 0.1, 0.2, 0.3, 0.4, 0.5, 0.6, 0.7, 0.8, 0.9, 1, 2 mm or 3 mm along its longest axis. In some embodiments, a drug implant can have a diameter of more than about 0.001, 0.01, 0.1, 0.2, 0.3, 0.4, 0.5, 0.6, 0.7, 0.8, 0.9, 1, 2, or 3 mm along its longest axis. A needle array head can be configured to accommodate at least about 1, 2, 3, 4, 5, 6, 7, 8, 9, 10, 20, 30, 40, 50, 60, 70, 80, 100, 200, 300, 400, or more than 1,000 drug implants. A needle array head can be configured to accommodate at most about 1, 2, 3, 4, 5, 6, 7, 8, 9, 10, 20, 30, 40, 50, 60, 70, 80, 100, 200, 300, 400, or more than 1,000 drug implants. A plurality of drug implants can be bundled to form a bundle of drug implants, wherein the bundle has a diameter suitable for injection using a needle.

An injection device (e.g., needle array device) can comprise a plunger configured to push one or more drug implants through needles into a solid tissue. Drug implants can be inserted along parallel axes in the solid tissue. Following insertion into a tumor, a drug agent can be released from one or more drug implants over a period of one minute to six weeks to allow discrete portions of the tumor to be exposed to each drug individually in a spatially confined manner.

An implant can be partially- or fully-submerged in the tissue. Deposition of implants into the tissue can be facilitated by inserting the implant into the tissue via a needle. The implant can be further deposited into the tissue by depression of a plunger associated with the needle.

Once an implant has been deposited into the tissue, the implant can dissolve and diffuse into the tissue. The rate of diffusion can be influenced by the volume of the implant, or it can be influenced by the presence of an inert binder that serves to reduce the rate of dissolution of the implant within the tissue. The drug agent can diffuse into the tissue over a period of about a minute to about a month; about an hour to about six weeks; about 12 hours to about 72 hours; about 24 hours to about 48 hours; about one week to two weeks; about two weeks to three weeks; or about three weeks to four weeks.

Microdialysis Probes

The present invention provides methods for the administration of an agent to a solid tissue through the use of one or more microdialysis probes. A microdialysis probe can be referred to as a diffusion device. In some cases, the microdialysis probe can comprise an inlet-tubing, an outlet-tubing and a membrane region. The solution in the inlet-tubing can be termed “perfusate” while the solution in the outlet tubing can be termed “dialysate”. The inlet- and outlet-tubings may be made of a material suitable for microdialysis application. In some embodiments, the material can be fused silica. In some instances, the microdialysis probe can comprise an inlet-tubing and a membrane region without an outlet-tubing. An agent may be actively pumped across the membrane region.

Microdialysis probes can be enclosed systems, not dependent upon delivery of a liquid volume, thus eliminating many of the microfluidic engineering hurdles. The semi-permeable membrane surrounding the probe can allow liquid to be filled and distributed evenly along probe membrane when injecting into a solid tissue. Initial delivery and biodistribution of agents can be highly restricted and dependent upon passive diffusion forces, not deposition and/or delivery of a liquid. “Microdosing” of agents can be achieved with microdialysis probes by controlling time, flow rate and concentration of perfusate. Multiple or timed dosing over an extended periods of time can be achieved by leaving probes in the solid tissue. The amount of agents delivered can be accurately determined by analyzing the amount of agent in perfusate and dialysate. The length of the probe/semi-permeable membrane can be customized to target various size tumors or length of targeting zone within a tumor. An array of linear microdialysis probes can be designed to target the proliferating zone in solid tumor xenografts, as well as avoiding the central regions necrosis. Better sampling of multiple zones, including the entire dimension of a solid tumor, to look for efficacy differences of agents using linear probe arrays can be achieved. Collection and analysis of dialysate at various time points following dosing may allow development and analysis of markers of tumor cell death, cell signal changes, or proliferation/mitotic changes. In addition, microdialysis probes can be used to induce contact-inhibited cells into cycling in order to kill them using checkpoint inhibition/DNA damage, or activate cell signal pathways that have been shut down in non-proliferative zones.

A microdialysis probe may be suitable for containing, administering, delivering, diffusing, and transporting agents. The agents within a single microdialysis probe may be the same or a mixture of different types of agents. Within a plurality of microdialysis probes, each microdialysis probe may contain the same agent as another probe, or different agents as another probe. In some embodiments, every microdialysis probe contains agents that are unique from the agents contained in other microdialysis probes. In some instances, some microdialysis probes comprise the same agents as in other microdialysis probes, and some microdialysis probes comprise different agents than in other microdialysis probes. In some instances, microdialysis probes can be delivered sequentially to a solid tissue. In some instances, microdialysis probes can be delivered simultaneously to a solid tissue. In some instances, multiple microdialysis probes can be delivered to the same site in a solid tissue.

A microdialysis probe may have a variety of different shapes. In some cases, the microdialysis probe can have a “Y” shape. In some other cases, the microdialysis probe can have a linear shape. The linear shape may allow the microdialysis probe to penetrate across different sections of a tumor.

The membrane of a microdialysis probe may be semi-permeable. The membrane may permit the transport of some but not all solutes. In some embodiments, the membrane can permit the transport of solutes with a molecule weight of less than 1 million daltons. In some embodiments, the membrane can permit the transport of solutes with a molecule weight in the range of 5,000 daltons to 1 million daltons. In some embodiments, the membrane can permit the transport of solutes with a molecule weight of less than 1,000 daltons.

The movement of a substance or an agent from one side to another side of a membrane may be driven by concentration gradient. In some cases, the movement of a substance or an agent from one side to another side of a membrane can be driven only by concentration gradient. A substance or an agent may move from an area of higher concentration to an area of lower concentration through the semi-permeable membrane. In some cases, the agent can diffuse from a microdialysis probe into a solid tissue. In some other cases, a solute (e.g., analyte) in a solid tissue can diffuse into a microdialysis probe. The solute can be collected and/or analyzed. The solute can be collected and/or analyzed from dialysate.

The movement of a substrate or an agent may be driven by active transporter, irrespective of concentration gradient. The movement of a substrate or an agent may be driven by solubility difference. The substrate or agent may have a higher solubility on one side of the membrane than the solubility on the other side. In some cases, the substrate or agent can move from a higher concentration side to a lower concentration side. In some embodiments, the substrate or agent can move from a lower concentration side to a higher concentration side. In some cases, the movement of a substance or an agent from one side to another side of a membrane can be driven by a combination of any one of concentration gradient, active transportation, and solubility difference.

The membrane may be biocompatible. The membrane may be essentially physiologically inactive or may not trigger physiological events. In some embodiments, the membrane may not cause inflammation, immune response, infection, or any other sort of rejections within a solid tissue. The membrane may be flexible. The flexibility of the membrane can permit the insertion of the membrane section into the solid tissue with minimal damage to the tissue. Yet, the membrane may have certain strength to maintain its integrity before, during or after the insertion. In some embodiments, the membrane can be both flexible and durable.

The membrane material may be polymeric or co-polymeric. The polymeric or co-polymeric material may be linear or cross-linked. Non-limiting examples of membrane materials include PE (polyethylene), Kevlar, cuprophane, polyethersulfone, polyamine, polyamide, polycarbonate, polycarbamate, polyurethane, polyester, polyether, polyolefin, polysilicon oxide, cellulose acetate, and polyaromatic materials.

The membrane material may be porous. In some embodiments, the average pore size can be less than about 1, 5, 10, 20, 30, 40, 50, 100, 200, 500, 1000, 2000, 5000, or 10000 nanometers. In some embodiments, the average pore size is more than about 1, 5, 10, 20, 30, 40, 50, 100, 200, 500, 1000, 2000, 5000, or 10000 nanometers. In some embodiments, the average pore size is in a range of 1-10, 1-40, 1-100, 1-200, or 1-500 nanometers. In some embodiments, all pores of a membrane have a substantially similar pore size. In some embodiments, some pores of a membrane have a substantially similar pore size and some have a different pore size. In some instances, pores of a membrane all have different pore sizes.

The pore size may control the rate of diffusion. The pore size may be modulated to control the rate of diffusion. A membrane may be made with a selected average pore size for the purpose of controlling the rate of diffusion. Different pharmaceutical compositions of agents can diffuse through the membrane at varying rates, controlled in part by the physical and chemical properties of the pharmaceutical compositions, agents, and membrane materials. In some embodiments, the selected pore size permits the transport of solutes with a molecule weight of less than 1 million daltons. The selected pore size may permit the transport of solutes with a molecule weight in the range of 5,000 daltons to 1 million daltons. The selected pore size may permit the transport of solutes with a molecule weight of less than 1,000 daltons. In addition, membranes with varying average pore sizes can be made and tested experimentally to find a pore size that provides a desirable diffusion rate for a specific pharmaceutical composition or agent.

A pharmaceutical composition or agent may be delivered to a microdialysis probe by using a pump, such as a peristaltic pump or syringe pump. The use of a pump can lead to controlled delivery. For example, the agent or pharmaceutical composition can be delivered through a microdialysis probe in a continuous fashion. Alternatively, the agent or pharmaceutical composition can be delivered in several doses. The time interval between any two doses can be controlled. Furthermore, the flow rate may be individually controlled for each microdialysis probe. The flow rate may be in a range of about 0.1 to about 5 microliter/minute. The flow rate may be at least about 0.1, 0.2, 0.3, 0.4, 0.5, 0.6, 1.0, 1.5, 2.0, 2.5, 3.0, 3.5, 4.0, 4.5, or about 5 microliter/minute. The flow rate may be at most about 0.1, 0.2, 0.3, 0.4, 0.5, 0.6, 1.0, 1.5, 2.0, 2.5, 3.0, 3.5, 4.0, 4.5, or about 5 microliter/minute.

A microdialysis probe may be inserted into a solid tissue directly or indirectly. The indirectly insertion may comprise the steps of: (1) insertion of a microdialysis probe into a needle; (2) insertion of the needle into a solid tissue; and (3) withdrawal of the needle from the solid tissue, therefore leaving the microdialysis probe in the solid tissue. In some cases, a plurality of microdialysis probes can be inserted into a solid tissue with a plurality of needles along a plurality of axes into a solid tissue. Each of the plurality of needles can hold one of the plurality of microdialysis probes. In some instances, some of the plurality of needles can hold some of the plurality of microdialysis probes. In some embodiments, the plurality of axes are a plurality of parallel axes. In some embodiments, the plurality of needles are part of an injection device (e.g., needle array device) of the disclosure.

In some embodiments, the microdialysis probe can comprise an inlet-tubing without an outlet-tubing. The terminal end of the probe can be surrounded by a semi-permeable membrane. The microdialysis probe may act as a diffuser in which liquid and small molecules are actively pumped or passively diffused across the semi-permeable membrane.

The insertion of a microdialysis probe may be guided. In some embodiments, the insertion of a microdialysis probe can be guided by a fixed guide to direct the insertion of a microdialysis probe into a selected region of a solid tissue. In some embodiments, the insertion of a microdialysis probe can be guided by an arthroscopic device.

The present disclosure also provides a method of monitoring drug metabolism and response in a solid tissue. For example, without being limiting, a microdialysis probe may be a part of closed loop. The membrane section of the microdialysis probe may span the solid tissue. By running a continuous flow of a solution of an agent through the microdialysis probe for a selected period of time, the agent may be delivered to the solid tissue. After another selected period of time, another solution (e.g. saline) may be flown through the microdialysis probe. Solutes in the solid tissue, for example without being limiting, may be collected in dialysate and analyzed. Non-limiting examples of solutes include biomarkers, agents delivered to the solid tissue and metabolites of the agents delivered to solid tissue. By analyzing the presence or absence and/or concentration of solutes, the efficacy of the agents on the solid tissue may be determined.

In some instances, the tube material can be coated with a substance that can prevent wound healing and/or clot formation. The coating can have antithrombogenic properties such as heparin.

Agents

An agent can be any fluid or molecule in an aqueous solution, mixture, or colloid that may be delivered to a target tissue. When used to refer to agent delivered through needles, the term agent can read on any substance capable of flowing through a microdialysis probe, drug implant, porous tube, and/or needle, such as liquids, gases, colloids, suspended solids, etc.

Agents can include, but are not limited to, a gene therapy agent, a chemotherapy agent, a small molecule, an antibody, a protein, a small interfering RNA, an antisense RNA, a ribozyme, a detectable label, a polypeptide, a peptidomimetic and an antibody-drug conjugate.

An agent can be a detectable label. A detectable label can include a radiolabel, a radio-opaque label, a fluorescent label, a colorimetric label, a dye, an enzymatic label, a GCMS tag, avidin, and biotin.

In some instances, two agents can refer to the same agent at different concentrations. For example, the prednisone at a concentration of 1 mM and at 10 mM can be two agents.

An agent can be a polynucleotide (e.g., a vector). A vector can comprise a polynucleotide sequence encoding an agent. The vector can comprise a promoter. The promoter may be operably linked to the polynucleotide of the vector. The promoter may be a constitutive promoter, an inducible promoter, and/or a tissue-specific promoter.

In some embodiments, the agents can be marketed anti-cancer drugs. Marketed anti-cancer drugs can include, but are limited to, Lomustine, Carmustine, Streptozocin, Mechlorethamine, Melphalan, Uracil Nitrogen Mustard, Chlorambucil, Cyclophosphamide, Iphosphamide, Cisplatin, Carboplatin, Mitomycin, Thiotepa, Dacarbazin, Procarbazine, Hexamethyl Melamine, Triethylene Melamine, Busulfan, Pipobroman, Mitotane, Methotrexate, Trimetrexate, Pentostatin, Cytarabine, Ara-CMP, Fludarabine phosphate, Hydroxyurea, Fluorouracil, Floxuridine, Chlorodeoxyadenosine, Gemcitabine, Thioguanine, 6-Mercaptopurine, Bleomycin, Topotecan, Irinotecan, Camptothecin sodium salt, Daunorubicin, Doxorubicin, Idarubicin, Mitoxantrone, Teniposide, Etoposide, Dactinomycin, Mithramycin, Vinblastine, Vincristine, Navelbine, Paclitaxel, Docetaxel. In some embodiments, the agents are candidate oncology agents. Candidate oncology agents can be selected from resources that disclose listings of investigational therapeutics, for instance, the National Institutes of Health (Bethesda, Md.) which maintains a database of ongoing and planned clinical trials at its “ClinicalTrials.gov” website.

In some embodiments, agents can be active forms of a prodrug. Prodrugs can be converted to their active form normally by natural metabolic processes. Prodrugs can be classified as Type I or Type II. Type I prodrugs are activated intracellulary. Type I prodrugs can include nucleoside analogs, idoxurine, 5-fluorouracil, 5-fluorocytosine, ganciclovir, Acyclovir, trifluorothymidine, adenine arabinoside bromovinyldeoxyuridine, penciclovir, diethylstilbestrol diphosphate, cyclophosphamide, L-dopa, 6-mercaptopurine, mitomycin C, zidovudine, Carbamazepine, captopril, carisoprodol, heroin, molsidomine, paliperidone, phenacetin, primidone, psilocybin, sulindac, fursultiamine, and MTX-α-peptide. Type II prodrugs are activated extracellularly. Type II prodrugs can include Lisdexamfetamine, loperamide oxide, oxyphenisatin, sulfasalazine, Acetylsalicylate, bacampicillin, bambuterol, chloramphenicol succinate, dihydropyridine pralidoxime, dipivefrin, and fosphenyloin. Chemotherapeutic prodrug therapy can include antibody-directed enzyme prodrug therapy (ADEPT), virus-directed enzyme prodrug therapy (VDEPT), gene-directed enzyme prodrug therapy (GDEPT), Clostridial-directed enzyme prodrug therapy (CDEPT). Prodrugs can be linked to nanoparticles or lipsosomes. Prodrugs can include R-CHOP drugs (i.e., rituximab, cyclophosphamide, doxorubicin, vincristine, and prednisolone). In some instances, an agent can be an active form of a pro-drug (e.g., not the prodrug itself but the active form that is activated once adminstered to a subject). For example, a prodrug can be prednisone, and the active form of the prodrug can be prednisolone. An agent can be prednisone or prednisolone.

In some embodiments, the agent can be a small molecule agent. As used herein, the term “small molecule agent” means an agent with a molecule weight less than about 1000 daltons, less than about 800 daltons, or less than about 500 daltons. In some embodiments, the small molecule agent can be an anti-cancer agent. The anti-cancer agent may be an approved anti-cancer drug currently on the market, an anti-cancer drug currently in clinical trials, an anti-cancer drug withdrawn from clinical trials or market, or an early stage anti-cancer drug in the development. An agent may be an agent for use in an investigational new drug trial.

Agents can be antibodies, including naturally occurring, immunologically elicited, chimeric, humanized, recombinant, and other engineered antigen-specific immunoglobulins and artificially generated antigen-binding fragments and derivatives thereof, such as single-chain antibodies, minibodies, Fab fragments, bi-specific antibodies and the like.

Agents can be antibody-drug conjugates. Antibody-drug conjugates (ADCs) can combine the binding specificity of monoclonal antibodies with the potency of chemotherapeutic agents. The technology can be associated with the development of monoclonal antibodies to tumor associated target molecules, the use of more effective cytotoxic agents, and the design of chemical linkers to covalently bind these components. An antibody-drug conjugate can comprise a linker between the monoclonal antibody and the cytotoxic agent. An antibody-drug conjugate can comprise non-native amino acids.

Agents can be agents that are active in ubiquitin pathways, and/or autophagy pathways. Agents can be proteasome inhibitors (e.g., bortezomib, ixazomib citrate). Agents can be a Bruton's tyrosin kinase (BTK) inhibitor (e.g, ibrutinib, GDC-0834, HM-71224, CC-292, and ONO-4059).

An agent can be an inhibitor or enhancer of the RAS/RAF/MEK, PI3K/AKT/mTOR, and/or JAK/STAT pathways.

A agent can be an inhibitor or enhancer of autophagy (e.g., N-acetyl-L-cysteine, L-asparagine, Bafilomycin, catalase, chloroquine diphosphate, DbeQ, E-64d, Leupeptin, 3-methyladenine autophagy inhibitor).

An agent can be a small molecule that modulates the immune system (e.g., lenalidomide, pomalidomide, analogs of thalidomide).

An agent can be provided as a member of a combinatorial library, which can include synthetic agents prepared according to a plurality of predetermined chemical reactions performed in a plurality of reaction vessels. For example, various starting compounds can be prepared employing solid-phase synthesis, recorded random mix methodologies and/or recorded reaction split techniques that permit a given constituent to traceably undergo a plurality of permutations and/or combinations of reaction conditions. The resulting products can comprise a library that can be screened followed by iterative selection and synthesis procedures, such as a synthetic combinatorial library of peptides or other compositions that can include small molecules.

An agent can comprise a pharmaceutical carrier. Pharmaceutically acceptable carriers can include sterile saline and phosphate-buffered saline at physiological pH, preservatives, stabilizers, and dyes. For example, sodium benzoate, sorbic acid and esters of p-hydroxybenzoic acid can be added as preservatives. In addition, antioxidants and suspending agents can be used. “Pharmaceutically acceptable salt” can refer to salts of drug compounds derived from the combination of such compounds and an organic or inorganic acid (acid addition salts) or an organic or inorganic base (base addition salts). The agents contemplated for use herein can be used in either the free base or salt forms, with both forms being considered as being within the scope of the certain present invention embodiments.

The pharmaceutical compositions that comprise one or more agents can be in any form which allows for the composition to be administered to a subject. Administration can include transcutaneous or subcutaneous injections, and intramuscular, intramedullar and intrastemal techniques.

The pharmaceutical composition can be formulated so as to allow the active ingredients contained therein to be bioavailable upon administration of the composition to a subject such as a human subject. Compositions that can be administered to a subject can take the form of one or more doses or dosage units. For example, a pre-measured fluid volume can comprise a single dosage unit. A device of the disclosure (e.g., needle, porous tube, drug implant, microdialysis probe) can hold a plurality of dosage units. A dose of an agent can include all or a portion of a therapeutically effective amount of a particular agent that is to be administered in a manner and over a time sufficient to attain or maintain a desired concentration range of the agent. The desired concentration range can be located in the immediate vicinity of a delivery microdialysis probe, needle, drug implant, and/or porous tube in a solid tissue. In some instances more than one dose can be administered to a subject. At least 2, 3, 4, 5, 6, 7, 8, 9, 10 or more doses can be administered. At most 2, 3, 4, 5, 6, 7, 8, 9, 10 or more doses can be administered. A dose or dosage unit can be a microdose.

An agent can comprise a liquid pharmaceutical composition. A liquid composition can comprise a solution and/or a suspension. A liquid composition can comprise an adjuvant. Adjuvants can include sterile diluents (e.g., water) for injection, physiological saline, Ringer's solution, saline solution (e.g., normal saline, or isotonic, hypotonic or hypertonic sodium chloride), fixed oils (e.g., synthetic mono or digylcerides), polyethylene glycols, glycerin, propylene glycol or other solvents, antibacterial agents (e.g, benzyl alcohol or methyl paraben), antioxidants (e.g., ascorbic acid or sodium bisulfite), chelating agents (e.g., ethylenediaminetetraacetic acid), buffers (e.g., acetates, citrates or phosphates and agents for the adjustment of tonicity such as sodium chloride or dextrose), stabilizers, and excipients.

The liquid composition can be enclosed in ampoules, disposable syringes or multiple dose vials made of glass or plastic, water-in-oil emulsions, biodegradable oil vehicles, oil-in-water emulsions, biodegradable microcapsules, hydrogels, and liposomes.

While any suitable carrier can be employed in the pharmaceutical compositions of the disclosure, the type of carrier can vary depending on the mode of administration and whether a conventional sustained drug release is also desired. For parenteral administration, such as supplemental injection of drug, the carrier can comprise water, saline, alcohol, a fat, a wax or a buffer.

A pharmaceutical carrier can comprise a nanosphere. The term “nanospheres” or “microsphere” can refer to carrier that are biocompatible with and sufficiently resistant to chemical and/or physical destruction by the environment of use such that a sufficient amount of the nanospheres and/or microspheres remain substantially intact after injection into solid tissue. A nanosphere can range in size from about 1 nm to about 1000 nm. The microspheres can range in size from about 1 micrometer to about 1000 micrometers. The agent can be loaded within and/or on the nanospheres/microspheres. The polymeric material that can be employed for forming the nanospheres and/or microspheres can include any biocompatible and biodegradable homopolymer or copolymer. Suitable examples of polymeric materials can include, but are not limited to: aliphatic polyesters, e.g., homopolymers or copolymers synthesized from one or more kinds of a-hydroxycarboxylic acids (e.g., glycolic acid, lactic acid, 2-hydroxybutyric acid, and valinic acid, leucic acid), hydroxydicarboxylic acids (e.g., malic acid), hydroxytricarboxylic acids (e.g., citric acid), or their mixtures; poly-α-cyanoacrylic esters, e.g., poly(methyl α-cyanoacrylate), poly(ethyl α-cyanoacrylate), poly(butyl α-cyanoacrylate); and amino acid polymers, e.g., poly(γ-benzyl-L-glutamate), or their mixtures. The mode of polymerization for these biodegradable polymers may be any of random, block or graft polymerization technique. In some instances, nanospheres can degrade. The degradation of the nanosphere can release the agent into the solid tissue.

Orientation

Because injected agents can be spatially defined in the tumor, tumor orientation can be important for evaluation of the agents. Loss of orientation can compromise the evaluation process because it can be unclear which injection location corresponded to which injected agent. Tumor orientation can be maintained by imaging the solid tissue and/or marking the solid tissue to show how the tumor was oriented during injection. Orientation can also be visually determined by injection of a visualizing agent. An agent can comprise a visualizing agent. A visualizing agent can comprise staining agents, inks, infrared dyes, fluorescent tattoo inks, Henna, india ink, new methylene blue, isosulfan blue dye, and rhodamina WT, colorimetric indicators, colorimetric pH indicators, reporter genes, chemically conjugated probes and fluorescent dyes. A portable light source may be used to detect solid tissue orientation and by identifying injected visualizing agents during surgery.

Position Markers

The disclosure provides for delivery of agents to a plurality of spatially defined locations along parallel axes in a solid tissue. In some instances, it can be important to define the position of one or more injected agents. In some instances, the injected agent can be a position marker. A position marker can comprise an agent that comprises a detectable source. For example, a position marker can comprise anything that can be detected (e.g., indicative of its location). A position marker can comprise nanoparticles, nanostructures, reporter molecules, dyes (e.g., Trypan blue, fluorescent dyes, radioactive dyes), a colorimetric pH indicator, a fluorescent compound that can exhibit distinct fluorescence as a function of any of a number of cellular physiological parameters (e.g., pH, intracellular Ca²⁺ or other physiologically relevant ion concentration, mitochondrial membrane potential, plasma membrane potential, etc.), an enzyme substrate, a specific oligonucleotide probe, a reporter gene, and the like.

Control Agents

In some instances, an agent can be a control agent. A control agent can be, for example, a negative control. A negative control can be a control that has been previously demonstrated to cause no statistically significant alteration of physiological state. Negative controls can include sham injections, saline, DMSO, inactive enantiomers, scrambled peptides or nucleotides.

In some instances a control agent can be a positive control. A positive control can be a control agent that has been previously demonstrated to cause a statistically significant alteration of physiological state.

Biomarkers

The present disclosure exemplifies a method for evaluating changes in the physiological status of solid tissues (e.g., tumor cells or tumorigenic cells) by measuring a biomarker. A biomarker can refer to any biological molecule (e.g., nucleic acid, polypeptide, small molecule, chemical, atom, ion) that can be indicative of a change in a cellular physiological process. In some instances, biomarkers can be secreted by cells. Secreted biomarkers can include autocrines, paracrines, or endocrines. In some instances, biomarkers can be measured in transcriptional levels as gene expressions or in protein levels. By measuring and detecting the biomarkers described herein over time, and relating the measurement to the biomarkers to a control sample, the physiological status or the changes in the physiological status of the tumor cells or tumorigenic cells, such as cell death, cell proliferation, cell signaling process or cellular responses, can be determined.

The death of tumor cells or tumorigenic cells can occur via apoptosis or necrosis. Apoptosis is a process of programmed cell death, and may be activated via either the death receptor-mediated extrinsic pathway or the mitochondria-directed intrinsic pathway. Non-limiting examples of biomarkers of apoptosis that can be measured in gene expressions or protein levels can include: activated caspase family such as caspases 2, 3, 7, 8, 9 and 10; tumor protein 53 (p53), phosphor-p53, p73, cyclin-dependent kinase inhibitor 1 (p21-waf1), and phosphor-H2AX/Ser 139 (pH2AX); B-cell lymphoma 2 (Bcl-2) family members such as Bcl-2, B-cell lymphoma-extra large (Bcl-XL), Bcl-xs, Bcl-W, and induced myeloid leukemia cell differentiation protein (Mcl-1); pro-apoptotic protein family such as Bcl-2-associated X protein (Bax), and Bcl-2 homologous antagonist/killer (Bak); Bcl-2 homology (BH) domain family such as BH1, BH2, BH3, BH4, Bcl-2-associated death promoter (Bad), p53 upregulated modulator of apoptosis (PUMA), NOXA, Bcl-2 modifying factor (Bmf), Bcl-2 interacting killer (Bik), Bcl-2-related ovarian killer (Bok), Bcl-2 interacting mediator of cell death (Bim), and BH3 interacting-domain death agonist (Bid); modulators of apoptosis proteins such as apoptotic protease activating factor 1 (APAF-1), apoptosis inducing factor (AIF), inhibitors of apoptosis (IAP) such as cIAP1, cIAP2, Cp-IAP, Op-IAP, XIAP, NAIP, survivin, and second mitochondria-derived activator of caspases (SMAC); markers to measure extent of DNA oxidative damage such as 8-hydroxy-2-deoxyguanosine and 3-nitrotyrosine; other biomarkers related to apoptosis such as cytochrome c, N-hydroxy-L-arginine (NOHA), 14-3-3 protein, tumor necrosis factor (TNF)-related apoptosis inducing ligand (TRAIL), reactive oxygen species (ROS), externalized phosphatidylserine, cytokeratins, poly(ADP-ribose) polymerase, nucleosomal DNA, apoptosis antigen 1 (Apo-1), TNF receptor superfamily, member 6 (Fas), Fas ligand (FasL), Fas-associated death domain protein (FADD), phosphorylated-FADD, glutathione-S-transferase-isoenzyme π (Gst-π), β-galactosidase, phosphorylated retinoblastoma suppressor protein and the like.

Necrosis can refer to premature death of cells or tissues, and may be caused by factors external to the cells or tissues. Other physiological events such as inflammatory responses of the cells may be triggered with necrosis. Non-limiting examples of biomarkers related to necrosis of tumor cells or tumorigenic cells that can be measured in gene expressions or protein levels can include tumor necrosis factor (TNF), cachexin, cachectin, lymphotoxin, cyclophilin A, interleukin-1, 2, 3, 4, 5, 6, 7, 8, 9, 10, 11, 12, 13, 14, 15, 16 and 17, alpha1-antitrypsin, copeptin, myeloperoxidase, FLICE-like inhibitory protein (FLIP), transducer and activator of transcription (STAT), tumor necrosis factor receptor superfamily, member 19 (TROY), cyclooxygenase (COX)-1, COX-2, cell death factors, macrophage inflammatory proteins, macrophage activating factors, macrophage migration inhibitory factors, neuroleukin, immunologic suppressor factors, transfer factors, oncostatin, osteopontin, interferon type I, interferon gamma, interleukin 1 receptor antagonist protein, CD70, CD30, CD40, 4-1BB ligand, ectodysplasins, B-cell activating factor, receptor activator of nuclear factor kappa-B ligand (RANKL), lymphotoxin and the like.

Biomarkers can relate to the proliferation/growth or mitotic activities of tumor cells or tumorigenic cells. Non-limiting examples of biomarkers can include Akt protein kinase B, Wilms tumor marker, retinoblastoma (Rb), Ki-67, proliferating cell nuclear antigen (PCNA), serine/threonine kinase, mammalian target of rapamycin (mTOR), neurotrophin, protein Mis18 beta, myostatin, cyclin dependent kinases (Cdk) 1, 2, 4, and 6, cyclin dependent kinase comples 2 (Cdc2 p34), cyclin D1, cyclin D2, cyclin D3, cyclin E, cyclin A, growth differentiation factors 1, 2, 3, 5, 6, 9, 10 and 15 and the like.

The physiological status of a cell may be heavily modulated by a plurality of signal transduction pathways. Signal transduction can occur when an extracellular signaling molecule or a ligand binds to and further activates a cell surface receptor, thereby altering intracellular molecules and creating a response. The biomarkers described herein can participate in the signaling pathways such as growth factors, enzymes, signaling factors, ligands, intermediate molecules generated in biological pathways, hormones, nutrients, transmembrane proteins, extracellular matrix proteins, intracellular components, downstream factors of protein phosphorylation and the like. Non-limiting examples of signal transduction biomarkers can include human epidermal growth factor receptor (HER) family molecules such as HER1, 3, and 4; phosphatidylinositol 3-kinases (PI3K)/protein kinase B (Akt) signaling pathway molecules as PI3K/AKT, microtubule-associated protein kinase (MAPK)/extracellular signal-regulated kinase (ERK) pathway molecules such as MAPK, mitogen-activated protein kinase (MEK), Ras, proto-oncogene serine/threonine-protein kinase (RAF), ERK1 and 2; hedgehog pathway proteins such as sonic hedgehog, desert hedgehog, indian hedgehog, hedgehog-interacting protein, smoothened protein (SMO), Gli-1, Gli-2, Gli-3, and forkhead box O (FoxO)-1; Wnt signal transduction pathway modulators such as Wnt1, 2, 2B, 3, 3A, 4, 5A, 5B, 6, 7A, 7B, 8A, 8B, 9A, 9B, 10A, 10B, 11, 16, Wnt1-inducible-signaling pathway protein 1 (Wisp-1), Wisp-2, and β-catenin (i.e. beta-catenin); parathyroid hormone-related proteins such as hypercalcemic hormone of malignancy, parathyroid hormone like tumor factor; phosphatase and tensin homolog (PTEN), serine/threonine-protein kinase (SGK3), eukaryotic translation initiation factor 4E-binding protein 1 (4E-BP1), tymidine kinase, growth hormone, pyruvate dehydrogenase lipoamide kinase isozyme 1 (PDK1), citrate, nitride oxide, P70S6 kinase, glycogen synthase kinase 3 (GSK-3), Src homology 2 domain containing (SHC)-transforming protein 1, CD117, platelet-derived growth factor receptor (PDGFR)-alpha, PDGFR-beta, vascular endothelial growth factor receptor-2 (VEGFR-2), epidermal growth factor receptor (EGFR), matrix metalloproteinase (MMP)-1, CD9, keratin 7, p27, parafibromin, BMI1 polycomb ring finger oncogene (Bmi-1), 14-3-3a, cystatin-SA, epididymal secretory protein E4, whey acidic protein (WAP) four-disulfide core domain protein 2 (WFDC2), adiponectin, leptin, resistin, agouti signaling protein, agouti-related protein, angiopoietins, angiostatic proteins, cysteine-rich protein 61, nephroblastoma overexpressed protein, peptide PHI, peptide YY, insulin, glucose, pituitary hormones, placental hormones, relaxin, secretin, urocortins, urotensins, vasoactive intestinal peptide, autocrine motility factor, beta-thromboglobulin, leukemia inhibitory factor, leukocyte migration-inhibitory factors, lymphotoxin-alpha, endothelin, enphrin, bradykinin, kininogens, tachykinins, chemokines such as chemokine C, CC, CXC, CX3C and the like.

A signal transduction pathway can be a ubiquitin pathway, and/or autophagy pathway.

A biomarker can be a growth factor. Non-limiting examples of growth factors that can be measured in gene expressions or protein levels to relate tumor cells or tumorigenic cells to a physiological status can include erythropoietin (EPO), angiopoietin (Ang), stem cell factor (SCF), vascular endothelial growth factor (VEGF), fibroblast growth factor (FGF), nerve growth factor (NGF), hematopoietic cell growth factor, hepatocyte growth factor, hepatoma-derived growth factor, migration-stimulating factor, autocrine motility factor, epidermal growth factor (EGF), insulin-like growth factor 1 (IGF-1), transforming growth factor (TGF), cartilage growth factor (CGF), keratinocyte growth factor (KGF), skeletal growth factor (SGF), osteoblast-derived growth factor (BDGF), cytoline growth factor (CGF), colony stimulating factor (CSF), integrin modulating factor (IMF), platelet-derived growth factor (PDGF), calmodulin, bone morphogenic proteins (BMP), tissue inhibitor matrix metalloproteinase (TIMP), and the like.

A biomarker can be a immunohistochemistry (IHC) marker. Non-limiting examples of IHC markers that can be measured can include hematopoetic markers, breast markers, carcinoma or mesothelial markers, colon markers, central nervous system markers, infectious disease markers, keratin or epithelial markers, lung markers, melanocytic markers, neuroendocrine markers/other hormones, other organ-related markers, prognostic other markers, prostate markers, stromal markers or tumor markers.

Hematopoetic biomarkers can include, but are not limited to: annexin A1, BCL2 follicular lymphoma marker, BCL6 follicle center B cell marker, CD10, CD20, CD23, CD79a, cyclin D1, hairy cell leukemia marker, multiple myeloma oncogene 1, PAX-g B cell transcriptional factor, ZAP 70, CD34, CD68, CD99, CD117, glycophorin-A, myeloperoxidase, terminal deoxynucleotidyl transferase, von willebrand factor VIII, anaplastic lymphoma kinase-1, CD15, CD30, fascin, CD45, CD138, kappa immunoglobulin light chains, lambda immunoglobulin light chains, plasma cell p63, CD1a, CD2, CD3, CD4, CD5, CD7, CD8, CD43, CD56, CD57 and granzyme B.

Breast biomarkers can include, but are not limited to: Akt protein kinase, cytokeratin 5, p63, epithelial antigen, cathepsin D, cytokeratin 8, HMW cytokeratin high molecule weight, cytokeratin 5/6, cytokeratin 7, cytokeratin 19, cytokeratin 20, E-cadherin, estrogen receptor, HER2/neu, Ki67 cell proliferation marker, p53 tumor suppressor gene protein, progesterone receptor and smooth muscle actin.

Carcinoma or medothelial biomarkers can include, but are not limited to: BER-EP4 epithelial antigen, calretinin, ERA epithelial related antigen, cervical or gynecological markers, p16 tumor suppressor gene protein, ProEx C biomarker, TAG72 and wilms tumor marker.

Colon biomarkers can include, but are not limited to: epidermal growth factor receptor, CDX2, microsatellite instability marker such as MLH1, MSH2, MSH6, PMS2 and p53.

Central nervous system (CNS) biomarkers can include, but are not limited to: human glial fibrillary acidic protein and neurofilament.

Infectious disease biomarkers can include, but are not limited to: cytomegalovirus, herpes simplex virus type I, II, pylori H and varicella zoster virus.

Keratin and epithelian biomarkers can include, but are not limited to: cytokeratin 5/6, cytokeratin 7, cytokeratin 8/18, cytokeratin 19, cytokeratin 20, cytokeratin high molecular weight, caldesmon smooth muscle, p63, collagen 9, smooth muscle myosin, cytokeratin cocktail and epithelial membrane antigen.

Lung biomarkers can include, but are not limited to: 34BE12, HMW cytokeratin high molecular weight, excision repair cross complementing polypeptide, synaptophysin and thyroid transcription factor-1.

Melanocytic biomarkers can include, but are not limited to: HMB melanoma associated marker 45, melanoma cocktail, melanoma associated marker 1, s100 protein and tyrosinase.

Neuroendocrine biomarkers and other hormones can include, but are not limited to: androgen receptor, calcitonin, chromogranin A, G cell antral pyloric mucosa, neuron-specific enolase, somatostatin and synaptophysin.

Other organ-related biomarkers can include, but are not limited to: CEA carcinoembryonic antigen, calectin-3, gross cyctic disease fluid protein 15, hepatocyte antigen, adrenal cortical inhibin and renal cell carcinoma marker.

Prostate biomarkers can include, but are not limited to: PIN2 cocktail, PIN4 cocktail, prostate specific antigen, prostatic acid phosphorase and p504s gene product.

Stromal markers can include, but are not limited to: CD31, podoplanin, DOG1 derived from GIST1, desmin filament protein, factor XIIIa fibrohistocytic, human herpesvirus type 8, muscle specific actin, myogenin muscle marker, myoglobin cardiac and skeletal marker, s100 protein, smooth muscle actin, smooth muscle myosin and vimentin.

Tumor markers can include, but are not limited to: alpha detoprotein, Ca 19-9 CI, Ca-125 epitheliod malign marker and survivin.

Biomarkers can be metabolites or metabolic biomarkers. Non-limiting examples of metabolites or metabolic biomarkers can include: adenosine triphosphate (ATP), adenosine diphosphate (ADP), adenosine monophosphate (AMP), cyclic adenosine monophosphate (cAMP), Guanosine-5′-triphosphate (GTP), Guanosine-5′-diphosphate (GDP), Guanosine-5′-monophosphate (GMP), nicotinamide adenine dinucleotide phosphate (NADP), NADPH, nicotinamide adenine dinucleotide (NAD), NADH, proliferating cell nuclear antigen, glucose, glucose-6-phosphate, fructose-6-phosphate, fructose 1,6-b phosphate, ribose-5-phosphate, erythrose-4-phosphate, xylulose 5-phosphate, glyceraldehyde-3-phosphate, sedoheptulose 7-phosphate, 3 ribulose-5-phosphate, 1 ribose-5-phosphate, phosphoenolpyruvate, 2-phosphoglycerate, 3-phosphoglycerate, 1, 3-phosphoglycerate, dihydroxyacetone phosphate, malate, oxaloacetate, ketoglutarate, lactate, glutamine, alanine, glutamate, pyruvate, fatty acids, acetyl-coA, citrate, glycerol, uric acid, cholesterols, eicosanoids, glycolipids, phospholipids, shpingolipids, steoid, triacylglycerols, albumin, insulin, diols, Ros, NO, bilirubin, phosphor-creatine, ketone bodies, L-ornithine, argininosuccinate, fumarate, L-arginine, urea, carbamoyl phosphate, ornithine, citrulline, histidine, isoleucine, leucine, lysine, methionine, phenylanine, threonine, tryptophan, valine, asparagines, aspartic acid, cysteine, glutamic acid, glycine, proline, selenocysteine, serine, taurine, tyrosine, citric acid and the like.

In some embodiments, the biomarkers can be ions. Non-limiting examples can include hydrogen, potassium, sodium, calcium, chloride, magnesium, bicarbonate, phosphate, hydroxyl, iodine, copper, iron, zinc, sulfate and the like.

The agent may comprise a plurality of agents. The plurality of agents may comprise at least one of a negative control composition. The plurality of agents may comprise at least one positive control composition. The plurality of agents may comprise at least one position marker. The plurality of agents may comprise at least one orientation marker. At least one of the plurality of agents may comprise an indicator of efficacy, which in certain further embodiments can comprise at least one of a nanoparticle, a nanostructure, and an indicator dye. At least one of the agents may be selected based on a clinically demonstrated efficacy of the respective agent. The methods of the disclosure may comprise assessing, with respect to at least one of the plurality of agents, at least one of efficacy, activity, and toxicity of the agent.

Target Tissues

Solid tissues may include any cohesive, spatially discrete non-fluid defined anatomic compartment that is substantially the product of multicellular, intercellular, tissue and/or organ architecture, such as a three-dimensionally defined compartment that may comprise or derive its structural integrity from associated connective tissue and may be separated from other body areas by a thin membrane (e.g., meningeal membrane, pericardial membrane, pleural membrane, mucosal membrane, basement membrane, omentum, organ-encapsulating membrane, or the like). Non-limiting exemplary solid tissues may include brain, liver, lung, kidney, prostate, ovary, spleen, lymph node (including tonsil), thyroid, pancreas, heart, skeletal muscle, intestine, larynx, esophagus and stomach.

In some instances, the tissue can be a soft tissue. Non-limiting examples of soft tissue can include muscle, adipose, skin, tendons, ligaments, blood, and nervous tissue.

In some embodiments, the tissue is, or is suspected of being, cancerous, inflamed, infected, atrophied, numb, in seizure, or coagulated. In some embodiments, the tissue is, or is suspected of being, cancerous. In some embodiments, the tissue is cancerous.

In some embodiments, the present method is directed to cancer, and the target tissue comprises a tumor, which may be benign or malignant, and can comprise at least one cancer cell. A cancer cell can include a prostate cancer cell, a breast cancer cell, a colon cancer cell, a lung cancer cell, a brain cancer cell, and an ovarian cancer cell, or any combination thereof. In certain embodiments, the tumor can comprise a cancer selected from lymphoma, adenoma, adenocarcinoma, squamous cell carcinoma, basal cell carcinoma, small cell carcinoma, large cell undifferentiated carcinoma, chondrosarcoma and fibrosarcoma.

In some embodiments, the tumor is resistant to a therapy, for example, a chemotherapy. The tumor may respond to the therapy initially but may develop resistance suddenly or gradually.

In some embodiments, the target tissue may not exhibit features of a disease, but may be used to assess the response of an individual tissue to one or more compounds.

Subjects

A solid tissue can be located in a subject. A subject can be a human subject, such as a patient that has been diagnosed as having or being at risk for developing or acquiring cancer. In some instances, a subject can be a human subject that is known to be free of a risk for having, developing or acquiring cancer. A human subject can be a subject involved in a clinical trial.

In some instances, a subject can be a non-human subject or biological source. Non-human subjects can include, for example, a non-human primate such as a macaque, chimpanzee, gorilla, vervet, orangutan, baboon or other non-human primate. A non-human subject can be preclinical models (e.g., model system), including preclinical models for solid tumors and/or other cancers. A non-human subject can be a mammal, for example, a mouse, rat, rabbit, pig, sheep, horse, bovine, goat, gerbil, hamster, guinea pig or any other mammal. The non-human subject can be a vertebrate, for example, a higher vertebrate, an avian, amphibian, or reptilian species.

A transgenic animal can refer to a non-human animal in which one or more of the cells of the animal includes a nucleic acid that is non-endogenous (i.e., heterologous). The non-endogenous nucleic acid can be present as an extrachromosomal element in a portion of its cell or stably integrated into its germ line DNA (i.e., in the genomic sequence of most or all of its cells).

Methods

The disclosure provides for methods for introducing a plurality of agents to a solid tissue and evaluating the plurality of agents using mass spectrometry. The plurality of agents can be introduced into the solid tissue by an injection device (e.g., needle array device) as described herein. Injection can occur at a plurality of sites (e.g., delivery sites, injection sites)

The introduced agents can be introduced continuously. The agents can be introduced continuously for at least 1 hour, 2 hours, 3 hours, 4 hours, 5 hours, 10 hours, 15 hours, 1 day, 2 days, 3 days, 4 days, 5 days, 6 days, 7 days, 2 weeks, 1 month or more. The agents can be introduced continuously for at most 1 hour, 2 hours, 3 hours, 4 hours, 5 hours, 10 hours, 15 hours, 1 day, 2 days, 3 days, 4 days, 5 days, 6 days, 7 days, 2 weeks, 1 month or more. In some instances, the agents can be introduced non-continuously (e.g., in discrete amounts, i.e., doses, e.g., microdoses).

In some instances, a first agent can be delivered systemically to a subject and a second agent can be delivered locally to a solid tissue within the subject. For example, a first agent can be delivered to the blood stream, and/or orally ingested, and a second agent can be injected into a solid tissue (e.g., using an injection device of the disclosure).

In some instances, a first agent can be delivered to a first site within a solid tissue and a second agent can be delivered to the same site in the solid tissue. In some instances, repetitive dosing can be performed. For example, the method of delivery can be repeated. For example, a method of delivery can refer to delivering one agent systemically and delivering another agent locally. A method of delivery can refer to injecting a first agent into a first site in a solid tissue and injecting a second agent into the same site. The method of delivery can be repeated at least 1, 2, 3, 4, 5, 6, 7, 8, 9, 10 or more times. The method of delivery can be repeated at least 1, 2, 3, 4, 5, 6, 7, 8, 9, 10 or more times.

After introduction of the agents into the target tissue, the target tissue can be evaluated after a selected period of time. A selected period of time can refer to a time in which an altered physiologic state occurs. A selected period of time can refer to a time in which no altered physiologic state has occurred. In some cases, one or more compounds may be administered to produce an altered physiologic state within a tissue.

A selected period of time can be at least 1 hour, 2 hours, 3 hours, 4 hours, 5 hours, 10 hours, 15 hours, 1 day, 2 days, 3 days, 4 days, 5 days, 6 days, 7 days, 2 weeks, 1 month or more. A selected period of time can be at most 1 hour, 2 hours, 3 hours, 4 hours, 5 hours, 10 hours, 15 hours, 1 day, 2 days, 3 days, 4 days, 5 days, 6 days, 7 days, 2 weeks, 1 month or more. After the selected period of time, the solid tissue may be evaluated. Evaluation can comprise removing the delivery sites of the plurality of agents in the solid tissue. Removal can occur by contact blotting, microdissecting, laser capture, and/or biopsy needle.

In some instances, a columnar region of a solid tissue can be removed. The columnar of the solid tissue can comprise the site of injection. The site of injection can refer to a length along an axis through the solid tissue where the injection device (e.g., needle) injected the agent. The columnar region to be removed can comprise the site of injection (e.g., can comprise the axial length of the site of injection through the tumor). In some instances, the columnar region may not comprise the site of injection. In some instances, an agent may permeate through the solid tissue. In some instances, the columnar region can comprise the region of permeation of the agent through the solid tissue.

In some embodiments, a solid tissue may be removed from a subject and frozen at a low temperature. The tissue can then be histologically sectioned. The plane of the section may be about orthogonal to the axis of the agents delivery. In some other embodiments, individual cells or cluster of cells are isolated by laser-capture microdissection or contact blotting of a tissue on a membrane target. In some other embodiment, tissue sample may be obtained by biopsy.

An excised solid tissue (e.g., comprising an agent, i.e., site of injection), can be prepared. Preparation (e.g., lysis) can occur by chemical (e.g., membrane-lysing agents) and/or mechanical means (e.g., sonication).

For example, sample preparation can occur by detergent extraction. Proteins can be solubilized using detergent extraction. A variety of detergents are available for protein extraction, including anionic, cationic, zwitterionic and non-ionic detergents. By virtue of their amphipathic nature, detergents can disrupt bipolar membranes to first free and then solublize proteins bound in the membrane or found inside the target cells. Exemplary anionic detergents can include chenodeoxycholic acid, N-lauroylsarconsine sodium salt, lithium dodecyl sulfate, 1-octanesulfonic acid sodium salt, sodium cholate hydrate, sodium deoxycholate, sodium dodecyl sulfate and glycodeoxycholic acid sodium salt. Cationic detergents can include cetylpyridinium chloride monohydrate and hexadecyltrimethylammonium bromide. Zwitterionic detergents can include CHAPS, CHAPSO, SB3-10 and SB3-12. Non-ionic detergents may be selected from N-decanoyl-N-methylglucamine, digitonin, n-dodecyl β-D-maltoside, octyl alpha-D-glucopyranoside, Triton X-100, Triton X-114, Tween 20 and Tween 80. A detergent can be a cleavable detergent, 3-[3-(1,1-bisalkyloxyethyl)pyridin-1-yl]propane-1-sulfonate (PPS). This detergent can be used to extract protein contained within the interior of the cell by disrupting cell membranes. Once the proteins are free from the cell, PPS can assist in protein solubilization by shielding the hydrophobic regions of the newly extracted protein from unfavorable interactions with water. The added advantage of PPS over conventional detergents such as sodium dodecyl sulfate or n-octylglucoside is that the detergent properties that interfere with MALDI mass spectrometry can be eliminated prior to analysis.

Sample preparation can occur with lipases. To the extent that lipid removal by detergent extraction is incomplete, one can choose to utilize enzymes to further degrade lipid contaminants. Such enzymes are called lipases, which can exhibit the catalytic triad Ser-Asp-His (an exception being geotrichium candidum which has Ser-Glu-His). Lipases can include triacylglycerol lipase (triglyceride lipase; tributyrase) phospholipase A2 (phosphatidylcholine 2-acylhydrolase, lecithinase A, phosphatidase, phosphatidolipase) lysophospholipase (lecithinase B, lysolecithinase, phospholipase B) acylglycerol lipase (monoacylglycerol lipase) galactolipase, phospholipase A1, lipoprotein lipase (clearing factor lipase, diglyceride lipase, diacylglycerol lipase) dihydrocoumarin lipase, 2-acetyl-1-alkylglycerophosphocholine esterase (1-alkyl-2-acetylglycerophosphocholine esterase, platelet-activating factor acetylhydrolase, PAF acetylhydrolase, PAF 2-acylhydrolase, LDL-associated phospholipase A2 LDL-PLA(2)), phosphatidylinositol deacylase (phosphatidylinositol phospholipase A2) phospholipase C (lipophosphodiesterase I, Lecithinase C, Clostridium welchii α-toxin, Clostridium oedematiens beta- and gamma-toxins) phospholipase D, (lipophosphodiesterase II, lecithinase D, choline phosphatase), phosphoinositide phospholipase C (triphosphoinositide phosphodiesterase, phosphoinositidase C, 1-phosphatidylinositol-4,5-bisphosphate phosphodiesterase, monophosphatidylinositol phosphodiesterase. phosphatidylinositol phospholipase C, PI-PLC, 1-phosphatidyl-D-myo-inositol-4,5-bisphosphate inositoltrisphosphohydrolase), alkylglycerophosphoethanolamine phosphodiesterase. (lysophospholipase D), glycosylphosphatidylinositol phospholipase D (GPI-PLD, glycoprotein phospholipase D, phosphatidylinositol phospholipase D, phosphatidylinositol-specific phospholipase D, phosphatidylinositol-glycan-specific phospholipase D), phosphatidylinositol diacylglycerol-lyase (1-phosphatidylinositol phosphodiesterase, monophosphatidylinositol phosphodiesterase, phosphatidylinositol phospholipase C, 1-phosphatidyl-D-myo-inositol inositolphosphohydrolase (cyclic-phosphate-forming)), glycosylphosphatidylinositol diacylglycerol-lyase ((glycosyl)phosphatidylinositol-specific phospholipase C, GPI-PLC, GPI-specific phospholipase C, VSG-lipase, glycosyl inositol phospholipid anchor-hydrolyzing enzyme, glycosylphosphatidylinositol-phospholipase C, glycosylphosphatidylinositol-specific phospholipase C, variant-surface-glycoprotein phospholipase C).

Sample preparation can occur with collagenases. Collagenases are enzymes that can cleave the peptide bonds in triple helical collagen molecules. Collagenase has been shown effective in isolating intact cells from a variety of tissues including bone, cartilage, thyroid, ovary, uterus, skin, endothelium, neuronal, pancreas, heart, liver and tumors.

Sample preparation can involve nucleic acid removal. Elimination of nucleic acids from sample prior to analysis can be achieved by chemical or enzymatic means. Chemical removal involves precipitation methods that employ polyethyleneimine (PEI) or streptomycin sulfate precipitation, followed by centrifugation. Alternatively, enzymes that specifically degrade DNA and/or RNA may be used to remove these molecules.

Sample preparation can include the use of protease inhibitors. In order to prevent proteins from being non-specifically degraded following extraction, it may be necessary to include inhibitors of proteases, which are enzymes that hydrolyze peptide bonds. Protease inhibitors can include antipain dihydrochloride (papain, trypsin); calpain inhibitor I (calpain I and II); calpain inhibitor II (calpain II and I); chymostatin (chymotrypsin); hirudin (thrombin); TLCK.HCl (trypsin, bromelain, ficin, papain); TPCK (chymotrypsin, bromelain, ficin, papain); and trypsin inhibitor (trypsin). Other inhibitors include APMSF, aprotinin, bestatin, leupeptin, pepstatin, PMSF, and TIMP-2.

In some instances, sample preparation can include proteolysis. Proteolysis can occur with enzymatic and chemical methods. Proteolysis agents can include trypsin and lysine specific proteinases, endoproteinase Glu-C, endoproteinase Asp-N, chymotrypsin, and cyanogen bromide.

The lysis can be purified. Purification can refer to techniques which can separate proteins and/or nucleic acids of interest from other proteins and/or nucleic acids. Purification techniques can include chromatography (e.g., ion exchange, affinity chromatography, size exclusion chromatography), ultracentrifugation, precipitation, dialysis, and gel purification (e.g., polyacrylamide gel purification). In some instances, the excised tissue is not lysed. The lysis can be further purified and/or prepared for mass spectrometry by, for instance, desalting the sample, or performing organic extraction

Once removed, the excised tissue can be cut into a plurality of serial histological sections along parallel planes that are substantially normal (e.g., perpendicular or deviating from perpendicular by as much as 1, 2, 3, 4, 5, 6, 7, 8, 9, 10, 11, 12, 13, 14, 15, 20, 25, 30, 35 or more degrees) to the parallel axes, for analysis by any of a number of known histological, histochemical, immunohistological, histopathologic, microscopic (including morphometric analysis and/or three-dimensional reconstruction), cytological, biochemical, pharmacological, molecular biological, immunochemical, imaging or other analytical techniques (e.g., mass spectrometry).

Mass Spectrometry

Mass spectrometer configurations and techniques can include matrix-assisted laser desorption/ionization source with a time-of-flight mass analyzer (MALDI-TOF), inductively coupled plasma-mass spectrometry (ICP-MS), accelerator mass spectrometry (AMS), thermal ionization-mass spectrometry (TIMS), quadrupole mass analyzers, ion trap analyzers, orbitrap analyzers, electrospray ionization mass spectrometry (ESI), fourier transform mass spectrometry (e.g., Fourier transform ion cyclotron resonance), tandem mass spectrometry (MS/MS), liquid chromatography mass spectrometry (LC/MS), and spark source mass spectrometry (SSMS). In some embodiments, a sample is analyzed with MALDI Imaging Mass Spectrometry.

Mass spectrometry techniques can be combined with other chromatographic techniques to evaluate agents. For example, mass spectrometry can be combined with liquid chromatography (e.g., HPLC), gas chromatography (e.g., GC/MS), and ion mobility (i.e., IMS/MS) wherein ions are separated under a gas.

Mass spectrometry techniques can also be combined with molecular biology techniques. For example, a protein may be excised from a polyacrylamide or agarose gel and subject to mass spectrometry. In some instances, the excised protein may be enzymatically digested (e.g., with trypsin, chymotrypsin).

Mass spectrometry can comprise generating ions or the sample and transporting the ions to the mass analyzer. Ionization can occur by electrons and/or chemical ionization. Ionization can be hard or soft (e.g., result in a high or low degree of sample fragmentation). Ionization techniques can include glow discharge, field desorption, fast atom bombardment, thermospray, desorption/ionization on silicon, direct analysis in real time (DART), atmospheric chemical ionization, secondary ion mass spectrometry, spark ionization, thermal ionization, and ion attachment ionization. Transporting can occur by magnetic or electric fields.

Mass spectrometry can be used to identify the contents of a sample (e.g., isotopic composition of a molecule in the sample), determine the structure of a molecule, determine the sequence of a polypeptide, determine the molecular weight of a molecule, obtain information about the isotopic abundance of elements in a molecule, obtain time-resolved data, and quantify the amount of a molecule and/or compound in a sample. Mass spectrometry can be used in pharmacokinetic studies. Mass spectrometry can have a very high sensitivity which can be useful for analyzing microdoses of agents (e.g., to tissues). Mass spectrometry can be used to identify modifications to biological molecules such as phosphorylation, ubiquitylation, methylation, acetylation, and glycosylation.

When the mass spectrometry to be used is MALDI-TOF, the prepared solid tissue sample may be mounted to a plate, such as a stainless steel plate. A solution of matrix may be coated to the sample. The solution of matrix may aid the ionization of various molecules in the tissue sample. The solution may comprise an organic acid (e.g., an acid in an organic solvent, e.g., acetonitrile, trifluoroacetic acid), for example, sinapinic acid, gentisic acid, ferulic acid, hydroxycinnamic acid, picolinic acid, dihydroxybenzoic acid, and methoxycinnamic acid. The sample may be dried prior to insertion of the plate in the mass spectrometer.

To carry out the mass spectrometry analysis by MALDI-TOF, a raster with laser spot may be irradiated on the surface of tissue. The laser spot may have a diameter in a range of about 10-100 micrometers. In some embodiments, the diameter is about 25 micrometers. The laser position may be fixed while the sample plate may be repositioned to obtain a mass image of the tissue sample. The laser may be fired at the sample continuously. The laser may be fired at the sample in pulses.

Mass spectrometry can be performed with electrospray mass spectrometry (ESI). ESI can produce gaseous ions from highly polar, mostly nonvolatile biomolecules, including lipids. The sample can be injected as a liquid at low flow rates (1-10 microliters/minute) through a capillary tube to which a strong electric field is applied. The field can generate additional charges to the liquid at the end of the capillary and produces a fine spray of highly charged droplets that are electrostatically attracted to the mass spectrometer inlet. The evaporation of the solvent from the surface of a droplet as it travels through the desolvation chamber can increase its charge density substantially. When this increase exceeds the Rayleigh stability limit, ions can be ejected and ready for MS analysis.

Mass spectrometry can be performed with ESI tandem mass spectrometry (ESI/MS/MS). ESI/MS/MS can simultaneously analyze both precursor ions and product ions, thereby monitoring a single precursor product reaction and producing (through selective reaction monitoring (SRM)) a signal only when the desired precursor ion is present. Complex mixtures such as crude extracts can be analyzed, but in some instances sample preparation is required.

In some instances, mass spectrometry can be performed with secondary ion mass spectrometry (SIMS). SIMS is an analytical method that can use ionized particles emitted from a surface for mass spectroscopy at a sensitivity of detection of a few parts per billion. The sample surface can be bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles.

In some instances, mass spectrometry can be performed with laser desorption mass spectroscopy (LD-MS). LD-MS can involve the use of a pulsed laser, which can induce desorption of sample material from a sample site—effectively, this means vaporization of sample off of the sample substrate. When coupled with Time-of-Flight (TOF) measurement, LD-MS can be referred to as LDLPMS (Laser Desorption Laser Photoionization Mass Spectroscopy). The LDLPMS method of analysis can give instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also can cause the molecules to break up into smaller fragment-ions. The positive or negative ions made are then accelerated into the flight tube, being detected at the end by a microchannel plate detector. Signal intensity, or peak height, can be measured as a function of travel time. The applied voltage and charge of the particular ion determines the kinetic energy, and separation of fragments are due to different size causing different velocity. Each ion mass will thus have a different flight-time to the detector.

After data collection on the mass spectrometer, the data can be analyzed using an atomic mass window of interest. For example, a window of interest can be defined from among the range of masses encompassed by the released proteins (the proteins released from the specimen due to the laser beam). This window of interest may include the entire range of masses of the released proteins or any portion thereof. Within the window of interest, masses of the released proteins may be analyzed so that the spatial arrangement of the released proteins may be determined. In other words, the location of released proteins may be correlated to their position (e.g., to the location of one or more sites) by reference to their measured mass within the window of interest. In some embodiments, this step may involve graphically depicting the mass of proteins within the atomic mass window of interest as a function of the linear distance between a first injection site and a second injection site (and/or other injection sites).

The analyzed proteins may be identified by reference to, for example, its mass number or other distinguishing characteristics. Identification can be facilitated by techniques such as extraction, HPLC fractionation, proteolysis, and/or mass spectrometric sequencing of one or more fragments and protein database searching.

A comparison may be made with data representing a normal tissue or sample, and/or comparison may be made with other types of data. For instance, comparison may be made with normal tissue or data associated with a normal tissue. In this manner, one may assess, for example, the state of disease progress and/or therapeutic treatment progress.

The data indicating the type of biomarkers present in the location of the tissue analyzed can be graphically depicted on an image of the solid tissue by plotting mass of the biomarkers along X and Y axes of the solid tissue. The biomarkers may be graphically depicted along Z axis (e.g. the axis for delivering the agents), thus providing three-dimensional graphs corresponding to the three-dimensional location in the solid tissue and across the entire solid tissue. For example, molecular images can be created from a raster over the surface of the sample with consecutive laser spots. Each spot (e.g., injection site) can produce a mass spectrum obtained from molecules present within the irradiated area. An MS image or molecular weight-specific map of the sample could be produced at any desired molecular weight value. The image or map of the sample can be overlayed on an image of the solid tissue, thereby correlating the contents of the irradiated sample in the mass spectrometer spot to the location of injection in the solid tumor. By analyzing a large number of areas surrounding the sites of injection in the solid tissue, a map of the biomarker distribution can be generated.

In some instances, the methods of the disclosure provide for using the mass spectrums generated from mass spectrometry to quantify an intensity of a biomarker and/or agent at a particular location in a solid tissue (e.g., site of injection, or area surrounding a site of injection). The intensity of the signal can correlate to or indicate the presence or absence of a biomarker and/or agent (e.g., whether the biomarker and/or agent is statistically significant above background levels). The intensity of the signal can correlate to or indicate the concentration of a biomarker and/or agent in a solid tissue.

The intensity data may be graphically depicted on an image of the solid tissue. The graphical depiction of the intensity of signal of the biomarker and/or agent can be thought of as a 4^{th} dimension of the three dimensional image of the solid tissue. Biomarker and/or agent localization can be plotted on the X and Y axes of the solid tissue (e.g., correlating to the location of injection and region of permeation of the agents). Biomarker and/or agent localization can be plotted along the Z axis, which can correspond to the columnar region along the axis of the site of injection. The intensity of the data (e.g., levels of the biomarker and/or agent) can also be extrapolated from the X, Y, and Z data, thereby generating a fourth dimension of data analysis of the solid tissue.

Through the use of the methods described herein, a large number of agents can be delivered to a solid tissue and their efficacies and/or toxicities can be compared. For example, if at least one biomarker for a process or a pathway is known, the abilities of multiple agents to inhibit or promote the process or pathway may be compared by analyzing the presence or absence, and the abundance or distribution of the at least one biomarker.

In some instances the methods of the disclosure provide for evaluating the rate of diffusion of an agent through the solid tissue. A rate of diffusion can be altered by the composition of the agent. For example, an agent comprising a hydrogel may diffuse at a slower rate than the same agent lacking the hydrogel.

The methods of the disclosure also provide for determining the dosages of an agent for systemic use in a subject. For example, the methods provide for determining the effective concentration of an agent necessary for inducing a therapeutic and/or physiological effect in a solid tissue. That concentration may be used to determine the systemic dosage needed to obtain the same concentration as if the agent were locally injected into the solid tissue.

In some instances, the methods provide for combining mass spectrums. Mass spectrums can be combined from each site of injection. Mass spectrums can be combined for repeated dosing at one site of injection.

In some instances, mass spectrums (or a combination of mass spectrums) can be compared to a reference mass spectrum in order to facilitate biomarker and/or agent identification and/or quantification.

In some instances, a combination of agents can be delivered to a first site, and one of the combination of agents can be delivered to a second site. The mass spectrums of the first site and the second site can be compared to evaluate the effect of the agent singly or in combination with other agents.

The present disclosure provides a method for identifying at least one biomarker of response for an agent (e.g., a biomarker indicative of the effect of an agent). For example, through the use of methods described herein, an agent and a control agent may be delivered to a solid tissue. The agent and the control agent can be delivered simultaneously to a solid tissue. In some embodiments, the agent and the control agent can be delivered sequentially to a solid tissue. Mass spectra at or near the sites of delivery for both the agent and control agent may be generated. By comparing the two spectra, a biomarker indicative of efficacy of the agent may be identified.

The present disclosure provides methods for evaluating the effect of two or more agents on a solid tissue. In some embodiments, the two or more agents can be delivered simultaneously to a same position of the solid tissue. In some embodiments, the two or more agents can be delivered sequentially to a same position of the solid tissue. After a selected period of time, a sample at or near the site of delivery can be prepared and analyzed by mass spectrometry. The mass spectra can be compared to determine relative efficacy and/or provide a comparison of the two or more agents.

In some embodiments, at least one agent is dosed systemically and one or more agents can be delivered to the solid tissue. After a selected period of time, a sample at or near the site of delivery is prepared and analyzed by mass spectrometry. Comparison of the two or more agents can be used to determine the effect of the two or more agents on each other and/or on an endogenous pathway and/or analyte. For example, the two or more agents may exhibit a synergistic effect or an additive effect. The method can compare the effect of a systemic dose versus a locally administered dose.

The present disclosure provides methods for determining an effective therapeutic concentration of an agent for treating a solid tissue, in particular, a tumor. For example, one or more agents may be delivered to a solid tissue at different concentrations. After a selected period of time, at least one sample containing one or more sites of delivery can be prepared and analyzed by mass spectrometry. Based on the analysis, such as the presence or absence or abundance and distribution of a specific biomarker, the effective therapeutic concentration of the agent at treating the solid tissue may be determined.

The present disclosure provides methods that are useful for the classification and/or stratification of a subject or subject population, including for use in drug discovery and in pharmacogenomics. For example, a subject can be classified for a clinical trial. A subject can be classified for any experimental trial.

Correlation of one or more biomarkers with a position at which one or more of a candidate agent has been introduced in a solid tumor can be used to gauge the subject's responsiveness to, or the potential efficacy of, a particular therapeutic treatment. Based on such correlation, multiple subjects may be grouped into different subgroups. This information may be used in future clinical trials for evaluation of tumor context dependent drug response.

In some instances, identification and comparison of biomarkers can be used to categorize the solid tissue in a subject. For example, the categorization can be based on genotype (e.g., as indicated by the absence or presence of a biomarker). The categorization can be used to categorize a solid tissue based on responsiveness to an agent (e.g., by measuring the agent and/or biomarker).

The categorization of a subject and/or a solid tissue can be used in making a subject-specific treatment decision. For example, subjects and/or solid tissues that are not deemed responsive to an agent may be candidates for treatment with another agent or combination of agents.

In some instances, the methods of the disclosure provide for evaluating cell lines and/or xenografts by mass spectrometry. For example, a cell line can be treated with an agent of the disclosure. After a selected period of time, the cells can be removed and prepared for mass spectrometry. In some instances, a xenograft of a tumor (e.g., a xenograft of a tumor placed into a model system, such as a mouse), can be injected with an agent. The xenograft can be removed and subjected to mass spectrometry. The agents and/or biomarkers identified from the mass spectrometry data can be graphically overlayed onto an image of the xenograft.

Computer Systems

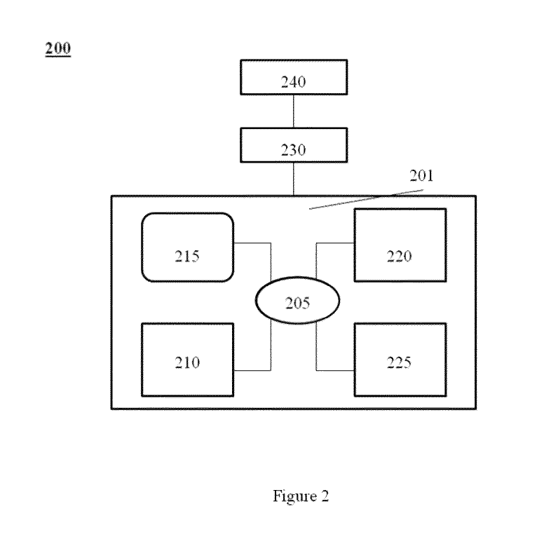

Another aspect of the invention provides a system that is configured to implement the methods of the disclosure. The system can include a computer server (“server”) that is programmed to implement the methods described herein. FIG. 2 depicts a system 200 adapted to enable a user to detect, analyze, and process mass spectrums and tumor images. The system 200 includes a central computer server 201 that is programmed to implement exemplary methods described herein. The server 201 includes a central processing unit (CPU, also “processor”) 205 which can be a single core processor, a multi core processor, or plurality of processors for parallel processing. The server 201 also includes memory 210 (e.g. random access memory, read-only memory, flash memory); electronic storage unit 215 (e.g. hard disk); communications interface 220 (e.g. network adaptor) for communicating with one or more other systems; and peripheral devices 325 which may include cache, other memory, data storage, and/or electronic display adaptors. The memory 210, storage unit 215, interface 220, and peripheral devices 225 are in communication with the processor 205 through a communications bus (solid lines), such as a motherboard. The storage unit 215 can be a data storage unit for storing data. The server 201 is operatively coupled to a computer network (“network”) 230 with the aid of the communications interface 220. The network 230 can be the Internet, an intranet and/or an extranet, an intranet and/or extranet that is in communication with the Internet, a telecommunication or data network. The network 230 in some cases, with the aid of the server 201, can implement a peer-to-peer network, which may enable devices coupled to the server 201 to behave as a client or a server. The insertion device and/or tumor imager can be peripheral devices 225 or remote computer systems 240.

The storage unit 215 can store files, such as images of tumors, data about tumor and/or tumor cells, mass spectrums, overlays of mass spectrums on tumor images, or any aspect of data associated with the invention.

The server can communicate with one or more remote computer systems through the network 230. The one or more remote computer systems may be, for example, personal computers, laptops, tablets, telephones, Smart phones, or personal digital assistants.

In some situations the system 200 includes a single server 201. In other situations, the system includes multiple servers in communication with one another through an intranet, extranet and/or the Internet.

The server 201 can be adapted to store tumor and mass spectrum information, such as, for example, tumor size, tumor morphology, shape, mass spectrums, concentrations and expression levels of biomarkers, and/or other information of potential relevance. Such information can be stored on the storage unit 215 or the server 201 and such data can be transmitted through a network.

Methods as described herein can be implemented by way of machine (or computer processor) executable code (or software) stored on an electronic storage location of the server 201, such as, for example, on the memory 210, or electronic storage unit 215. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210. Alternatively, the code can be executed on a second computer system 240.

Aspects of the systems and methods provided herein, such as the server 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

The results of tumor insertion and mass spectrum analysis can be presented to a user with the aid of a user interface, such as a graphical user interface.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Evaluation of Agents with Mass Spectrometry

In some embodiments of the methods of the disclosure, an agent is loaded into an injection device. The agents loaded into the injection device comprise an anti-cancer agent and a control agent. The agents are injected into the solid tissue, resulting in a plurality of delivery sites. The delivery sites comprise the agents, wherein one delivery site comprises a first agent; a second delivery site comprises a second agent, etc.

After 24 hours, the solid tissue is imaged. The injection sites are removed and lysed. The lysis is desalted. The lysis is mixed with a matrix and spotted on a MALDI-TOF analysis plate. The spots are dried and then subjected to imaging mass spectrometry.

The mass spectrometry data is compiled and overlayed on the image of the tumor. In some instances, the mass spectrometry data indicates the abundance, distribution and/or presence or absence of an agent and/or a biomarker. In some instances, the agent and/or biomarker is more densely localized at or near the sites of injection. The agent and/or biomarker is less densely localized further from the injection site.

### Example 2

### Categorization of Subjects by Evaluation of Solid Tissues with Mass Spectrometry

In some embodiments of the methods of the disclosure, an agent is loaded into an injection device. The agents loaded into the injection device comprise an anti-cancer agent and a control agent. The agents are injected into the solid tissue, resulting in a plurality of delivery sites. The delivery sites comprise the agents, wherein one delivery site comprises a first agent, a second delivery site comprises a second agent, etc.

After 24 hours, the solid tissue is imaged. The injection sites are removed and lysed. The lysis is desalted. The lysis is mixed with a matrix and spotted on a MALDI-TOF analysis plate. The spots are dried and then subjected to imaging mass spectrometry.

The mass spectrometry data is compiled and the solid tissue is evaluated for response to the agent. In some instances, the solid tissue is found to be unresponsive to the agent. The solid tissue and/or the subject from which the solid tissue originated is deemed acceptable for a clinical trial of an alternative agent. Alternatively, the solid tissue and/or the subject from which the solid tissue originated is deemed acceptable for a different therapeutic regimen which is specific to the physiological makeup of the solid tissue.

1-130. (canceled)

131. A method for evaluating an agent in a solid tissue of a subject, comprising:
(a) delivering two or more agents to two or more sites in said solid tissue using an administration device, wherein said administration device comprises two or more needles in an array, wherein a first needle of said two or more needles comprises a first agent of said two or more agents and a second needle of said two or more needles comprises a second agent of said two or more agents;
(b) performing mass spectrometry on a sample comprising at least one of said two or more sites; and
(c) evaluating said two or more agents for an effect on said tissue.

132. The method of claim 131, wherein said administration device is configured to deliver said two or more agents at an amount undetectable outside said solid tissue,

133. The method of claim 131, wherein said administration device is configured to deliver said two or more agents at a therapeutically effective amount.

134. The method of claim 131, wherein said two or more agents are delivered at or below a systemically detectable concentration.

135. The method of claim 131, wherein said delivering is performed in vivo.

136. The method of claim 131, wherein said delivering is performed in vitro.

137. The method of claim 131, wherein a region of permeation of said first agent in said solid tissue is separate from a region of permeation of said second agent in said solid tissue.

138. The method of claim 131, wherein an amount of said two or more agents delivered to said solid tissue is less than 5 microliters per needle.

139. The method of claim 131, wherein said administration device is configured for passive delivery of said two or more agents into said solid tissue.

140. The method of claim 131, wherein said two or more agents are delivered simultaneously to said solid tissue.

141. The method of claim 131, wherein said two or more agents are delivered sequentially to said solid tissue.

142. The method of claim 131, wherein said administration device comprises 6 or more needles.

143. The method of claim 131, further comprising removing said two or more sites.

144. The method of claim 143, where said removing comprises removing by biopsy needle.

145. The method of claim 143, wherein said removing comprises removing said two or more sites by skin punch biopsy.

146. The method of claim 143, wherein said removing removes a columnar region of said solid tissue comprising said site.

147. The method of claim 143, wherein said removing removes an area adjacent to said site.

148. The method of claim 131, wherein said mass spectrometry is imaging mass spectroscopy.

149. The method of claim 131, wherein said mass spectrometry is tandem MS/MS.

150. The method of claim 131, wherein said mass spectrometry is Matrix-Assisted Laser Desorption Ionization Mass Spectrometry (MALDI MS).

151. The method of claim 131, wherein at least one of said two or more agents comprises a position marker.

152. The method of claim 131, wherein at least one of said two or more agents is selected from the group consisting of: a negative control, a positive control, an agent in an investigative new drug trial, an agent in the orange book, an agent in a clinical trial, a biologic, a gene therapy agent, an antibody-drug conjugate, a chemotherapeutic agent, a small-molecule, an anti-cancer agent, and an agent that interferes with RNA activity, or any combination thereof.

153. The method of claim 131, wherein said two or more agents comprises 10 or more agents.

154. The method of claim 131, wherein said solid tissue comprises a tumor.

155. The method of claim 131, wherein said solid tissue comprises a skin-related tumor.

156. The method of claim 131, wherein said solid tissue comprises lymphoma.

157. The method of claim 131, wherein said performing mass spectrometry comprises analyzing an atomic mass window.

158. The method of claim 157, wherein said analyzing said atomic mass window comprises graphically depicting a mass of a biomarker along an axis of said two or more sites.

159. The method of claim 157, wherein said analyzing said atomic mass window comprises graphically depicting a mass of a biomarker along an axis of said solid tissue selected from the group consisting of: X, Y, and Z, or any combination thereof.

160. The method of claim 159, further comprising quantifying an intensity of a signal of one or more mass spectrums of said biomarker.

161. The method of claim 160, wherein said intensity correlates to a presence or absence of said biomarker.

162. The method of claim 160, wherein said intensity correlates to a concentration of said biomarker.

163. The method of claim 131, wherein said evaluating comprises determining a rate of diffusion of at least one of said two or more agents.

164. The method of claim 131, wherein said evaluating comprises determining a dosage for systemic administration of at least one of said two or more agents.

165. The method of claim 131, wherein said evaluating comprises determining a physiological response.

166. The method of claim 131, wherein said evaluating comprises pooling mass spectrums.

167. The method of claim 131, wherein said evaluating comprises detecting a biological modification.

168. The method of claim 167, wherein said biological modification comprises a modification to a polypeptide.

169. The method of claim 167, said biological modification comprises a modification to a nucleic acid.

170. The method of claim 131, wherein said evaluating comprises detecting a concentration of a biomarker.

171. The method of claim 131, wherein said evaluating comprises detecting a presence or absence of a biomarker.

172. The method of claim 131, wherein said evaluating comprises mapping a distribution of a biomarker on an image of said solid tissue.

173. The method of claim 131, wherein said evaluating comprises detecting a degree of permeation of at least one of said two or more agents through said solid tissue.

174. The method of claim 131, wherein said evaluating comprises comparing an activity said first agent with said second agent on said solid tissue.

175. The method of claim 131, wherein said evaluating comprises assessing a toxicity of said two or more agents on said solid tissue.

176. The method of claim 131, wherein said evaluating comprises comparing a mass spectrum of said two or more sites with a reference mass spectrum.

177. The method of claim 131, wherein a first site of said one or more sites comprises an agent and a second site of said one or more sites comprises a control agent.

178. The method of claim 177, wherein said evaluating comprises comparing a mass spectrum of said first site with a mass spectrum of said second site.

179. The method of claim 131, further comprising administering a treatment regimen specific to said genotype.

180. A system for implementing the method of claim 131, comprising:
(a) a storage memory for storing a dataset associated with a sample;
(b) a processor communicatively coupled to the storage memory; and
(c) a dataset.

181. A method for evaluating a combination of agents in a solid tissue of a subject, comprising:
(a) delivering a combination of two agents to a first site in said solid tissue using an administration device, wherein said administration device comprises two or more needles, wherein a first needle of said two or more needles comprises a first agent and a second needle of said two or more needles comprises a second agent;
(b) delivering one of said two agents to a second site in said solid tissue; and
(c) comparing said first site with said second site,
wherein said comparing comprises comparing said agents for a physiological effect.

182. A method for evaluating an agent in a solid tissue of a subject, comprising:
(a) delivering a first agent to a site in said solid tissue using an administration device, wherein said administration device comprises two or more needles in an array;
(b) delivering a second agent systemically to a subject;
(c) evaluating said first agent for a physiological effect using mass spectrometry.

183. A method for evaluating an agent in a solid tissue of a subject, comprising:
(a) delivering two or more agents to two or more sites in said solid tissue using an administration device, wherein said administration device comprises two or more microdialysis probes, wherein a first microdialysis probe of said two or more microdialysis probes comprises a first agent of said two or more agents and a second microdialysis probe of said two or more microdialysis probes comprises a second agent of said two or more agents;
(b) performing mass spectrometry on a sample comprising said two or more sites; and
(c) evaluating said two or more agents for efficacy.
